(19) United States
(12) Patent Application Publication
(10) Pub. No.: US 2026/0234128 A1
(43) Pub. Date: Aug. 13, 2026

(54) CRYSTALLINE FORMS OF 2-(4-(4-(AMINOMETHYL)-1-OXO-1,2- DIHYDROPHTHALAZIN-6-YL)-1-METHYL-1H- PYRAZOL-5-YL)-4-CHLORO-6-CYCLOPROPOXY-3-FLUOROBENZONITRILE

(71) Applicant: MIRATI THERAPEUTICS, INC., PRINCETON, NJ (US)

(72) Inventors: MICHAL ACHMATOWICZ, SAN DIEGO, CA (US); SVITLANA KULYK, SAN DIEGO, CA (US); CHRISTOPHER RONALD SMITH, SAN DIEGO, CA (US); NAN WANG, SOUTH PLAINFIELD, NJ (US); HARSH SHAH, SOUTH PLAINFIELD, NJ (US); TIAN XIE, SOUTH PLAINFIELD, NJ (US); QI GAO, SOUTH PLAINFIELD, NJ (US); NIKHIL LOKA, ESCONDIDO, CA (US)

(73) Assignee: MIRATI THERAPEUTICS, INC., PRINCETON, NJ (US)

(21) Appl. No.: 19/155,571

(22) PCT Filed: Feb. 12, 2024
(86) PCT No.: PCT/US2024/015341

§ 371 Date: Aug. 12, 2025

Related U.S. Application Data
(60) Provisional application No. 63/484,602, filed on Feb. 13, 2023.
(60) Provisional application No. 63/516,668, filed on Jul. 31, 2023.

(51) Int. Cl. C07D 403/04; A61K 31/502

(52) U.S. Cl. CPC C07D 403/04; A61K 31/502

(57) ABSTRACT

The present invention relates to crystalline forms of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile hydrochloride salt, pharmaceutically acceptable compositions comprising these crystalline forms, and methods for using these crystalline forms.

### CROSS-REFERENCE TO RELATED APPLICATIONS

This application claims priority from U.S. Provisional Application No. 63/484,602, filed Feb. 13, 2023, and U.S. Provisional Application No. 63/516,668, filed Jul. 31, 2023, the disclosure of each of which is hereby incorporated by reference in its entirety.

### FIELD OF THE INVENTION

This invention relates to hydrochloride salts of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile and particular crystalline forms thereof, processes of preparing the crystalline forms, and pharmaceutical compositions including the crystalline forms. The crystalline forms thereof are useful in the treatment and/or prevention of diseases and/or conditions related to cell proliferation, such as cancer. In particular, the crystalline forms provide therapeutic benefits as MTA-cooperative inhibitors of Protein Arginine N-Methyl Transferase 5 (PRMT5).

### BACKGROUND OF THE INVENTION

Protein Arginine N-Methyl Transferase (PRMT5) is a Form II arginine methyltransferase that catalyzes the transfer of a methyl group from S-adenosyl-L-methionine (SAM) to an omega-nitrogen of the guanidino function of protein L-arginine residues (omega-monomethylation) and the transfer of a second methyl group to the other omega-nitrogen, yielding symmetric dimethylarginine (sDMA). PRMT5 forms a complex with MEP50 (methylosome protein 50), which is required for substrate recoginition and orientation and is also required for PRMT5-catalyzed histone 2A and histone 4 methyltransferase activity (e.g., see Ho et al., (2013) PLOS ONE 8(8): 10.1371/annotation/e6b5348e-9052-44ab-8f06-90d01dc88fc2).

Homozygous deletions of p16/CDKN2a are prevalent in cancer and these mutations commonly involve the co-deletion of adjacent genes, including the gene encoding methylthioadenosine phosphorylase (MTAP). It is estimated that approximately 15% of all human cancers have a homozygous deletion of the MTAP gene (e.g., see Firestone & Schramm (2017) J. Am. Chem Soc. 139(39):13754-13760. doi: 10.1021/jacs.7b05803. Epub 2017 Sep. 20).

Cells lacking MTAP activity have elevated levels of the MTAP substrate, methylthioadenosine (MTA), which is a potent inhibitor of PRMT5. Inhibition of PRMT5 activity results in reduced methylation activity and increased sensitivity of cellular proliferation to PRMT5 depletion or loss of activity. Hence, the loss of MTAP activity reduces methylation activity of PRMT5 making the cells selectively dependent on PRMT5 activity.

Thus, MTA-cooperative inhibition of PRMT5 activity in MTAP deleted cancers can provide therapeutic benefit for a wide range of cancers. The compounds of the invention provide this therapeutic benefit as MTA-cooperative inhibitors of PRMT5 that negatively modulate the activity of MTA-bound PRMT5 in a cell, particularly an MTAP-deficient cell, or for treating various forms of MTAP-associated cancer.

In particular, 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile, is a potent and selective inhibitors of PRMT5 and has been found to be pharmacologically active. Here, crystalline hydrochloride salts of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile have been found that are suitable for use in pharmaceutical compositions.

### SUMMARY OF THE INVENTION

In one aspect, this disclosure provide crystalline forms of the hydrochloride salt of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile, shown below, and hereinafter Compound 1.

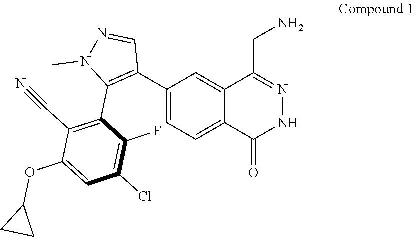

In another aspect, this disclosure also provides a particular crystalline form of Compound 1, hereinafter hydrochloride (HCl) salt Form A. This disclosure further provides processes of preparing HCl salt Form A. This disclosure further provides pharmaceutical compositions comprising HCl salt Form A and a pharmaceutically acceptable carrier.

In another aspect, this disclosure also provides a particular crystalline form of Compound 1, hereinafter hydrochloride (HCl) salt Form B. This disclosure further provides processes of preparing HCl salt Form B. This disclosure further provides pharmaceutical compositions comprising HCl salt Form B and a pharmaceutically acceptable carrier.

In another aspect, this disclosure also provides a particular crystalline form of Compound 1, hereinafter hydrochloride (HCl) salt Form E. This disclosure further provides processes of preparing HCl salt Form E. This disclosure further provides pharmaceutical compositions comprising HCl salt Form E and a pharmaceutically acceptable carrier.

In another aspect, this disclosure also provides a particular crystalline form of Compound 1, hereinafter hydrochloride (HCl) salt Form F. This disclosure further provides processes of preparing HCl salt Form F. This disclosure further provides pharmaceutical compositions comprising HCl salt Form F and a pharmaceutically acceptable carrier.

In another aspect, the present disclosure also provides a particular crystalline form of Compound 1, herein after hydrochloride (HCl) salt Form G. The present disclosure further provides processes of preparing HCl salt Form G. The present disclosure further provides pharmaceutical compositions comprising HCl salt Form G and a pharmaceutically acceptable carrier.

This disclosure further provides methods of treating cancer comprising administering to a subject in need of such treatment a crystalline form of Compound 1 as disclosed herein or a pharmaceutical composition comprising a crystalline form of Compound 1.

### BRIEF DESCRIPTION OF THE FIGURES

FIG. 1A is a differential scanning calorimetric (DSC) thermogram and a thermal gravimetric analysis (TGA) overlay of a HCl Form A crystal of Compound 1.

FIG. 1B is an X-ray powder diffraction (XRPD) pattern of a HCl Form A crystal of Compound 1.

FIG. 1C is a polarized light microscopy (PLM) image of a HCl Form A crystal of Compound 1.

FIG. 1D is a dynamic vapor sorption (DVS) isotherm plot of a HCl Form A crystal of Compound 1.

FIG. 2A is a differential scanning calorimetric (DSC) thermogram and a thermal gravimetric analysis (TGA) overlay of a HCl Form B crystal of Compound 1.

FIG. 2B is an X-ray powder diffraction (XRPD) pattern of a HCl Form B crystal of Compound 1.

FIG. 2C is a polarized light microscopy (PLM) image of a HCl Form B crystal of Compound 1.

FIG. 2D is a dynamic vapor sorption (DVS) isotherm plot of a HCl Form B crystal of Compound 1.

FIG. 3A is a differential scanning calorimetric (DSC) thermogram and a thermal gravimetric analysis (TGA) overlay of a HCl Form E crystal of Compound 1.

FIG. 3B is an X-ray powder diffraction (XRPD) pattern of a HCl Form E crystal of Compound 1.

FIG. 3C is a dynamic vapor sorption (DVS) isotherm plot of a HCl Form E crystal of Compound 1.

FIG. 3D is a proton nuclear magnetic resonance (¹H-NMR) spectra of a HCl Form E crystal of Compound 1.

FIG. 4A is a differential scanning calorimetric (DSC) thermogram and a thermal gravimetric analysis (TGA) overlay of a HCl Form F crystal of Compound 1.

FIG. 4B is an X-ray powder diffraction (XRPD) pattern of a HCl Form F crystal of Compound 1.

FIG. 4C is a proton nuclear magnetic resonance (¹H-NMR) spectra of a HCl Form F crystal of Compound 1.

FIG. 5A is a differential scanning calorimetric (DSC) thermogram and a thermal gravimetric analysis (TGA) overlay of a HCl Form G crystal of Compound 1.

FIG. 5B is an X-ray powder diffraction (XRPD) pattern of a HCl Form G crystal of Compound 1.

FIG. 5C is a proton nuclear magnetic resonance (¹H-NMR) spectra of a HCl Form G crystal of Compound 1.

FIG. 6A is an X-ray powder diffraction (XRPD) pattern of an amorphous HCl salt of Compound 1.

FIG. 6B is a polarized light microscopy (PLM) image of an amorphous HCl salt of Compound 1.

FIG. 6C is a differential scanning calorimetric (DSC) thermogram and a thermal gravimetric analysis (TGA) overlay of an amorphous HCl salt of Compound 1.

FIG. 6D is a proton nuclear magnetic resonance (¹H-NMR) spectra of an amorphous HCl salt of Compound 1.

FIG. 6E is an high pressure liquid chromatography (HPLC) spectra of an amorphous HCl salt of Compound 1.

FIG. 7A is a graph area under the curve for plasma concentrations for HCl Form A of Compound 1 and HCl Form B of Compound 1 administered in mammals.

FIG. 7B is a graph area under the curve for plasma concentrations for HCl Form B of Compound 1 administered in mammals.

FIG. 8 is a graph of the total impurity content vs. excipient for HCl Form B of Compound 1.

FIG. 9A is a graph of the porosity vs. compaction pressure for HCl Form B of Compound 1.

FIG. 9B is a graph of the tensile strength vs. compaction pressure for HCl Form B of Compound 1.

FIG. 9C is a graph of the tensile strength vs. porosity for HCl Form B of Compound 1.

FIG. 10 is an X-ray powder diffraction (XRPD) pattern of an HCl Form B crystal of Compound 1 before and after compression at high force.

FIG. 11A is a graph of the intrinsic dissolution rate of HCl Form B of Compound 1.

FIG. 11B is a graph of the dissolution of HCl Form B of Compound 1 in a capsule in biorelevant media.

### DETAILED DESCRIPTION OF THE INVENTION

As noted above, the invention provides particular crystalline forms of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile hydrochloride salt.

In some embodiments, the invention also provides HCl salt Form A, i.e., a particular crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile hydrochloride salt.

In some embodiments, the invention also provides HCl salt Form B, i.e., a particular crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile hydrochloride salt.

In some embodiments, the invention also provides HCl salt Form E, i.e., a particular crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile hydrochloride salt.

In some embodiments, the invention also provides HCl salt Form F, i.e., a particular crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile hydrochloride salt.

In some embodiments, the invention also provides HCl salt Form G, i.e., a particular crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile hydrochloride salt.

The crystalline forms as described herein may be characterized using a number of methods known to the person of ordinary skill in the art including thermal analysis (e.g., differential scanning calorimetry (DSC), thermal gravimetric analysis (TGA)), X-ray powder diffraction (XRPD), microscopy (e.g., scanning electron microscopy (SEM), polarized light microscopy), and spectroscopy (e.g., infrared, Raman, solid-sate nuclear magnetic resonance, proton nuclear magnetic resonance (¹HNMR)). The particle size and size distribution may be determined by conventional methods, such as laser light scattering technique. The purity of the crystalline forms provided herein may be determined by standard analytical methods, such as thin layer chromatography (TLC), gel electrophoresis, gas chromatography, high performance liquid chromatography (HPLC), and mass spectroscopy (MS).

### HCl Salt Form A

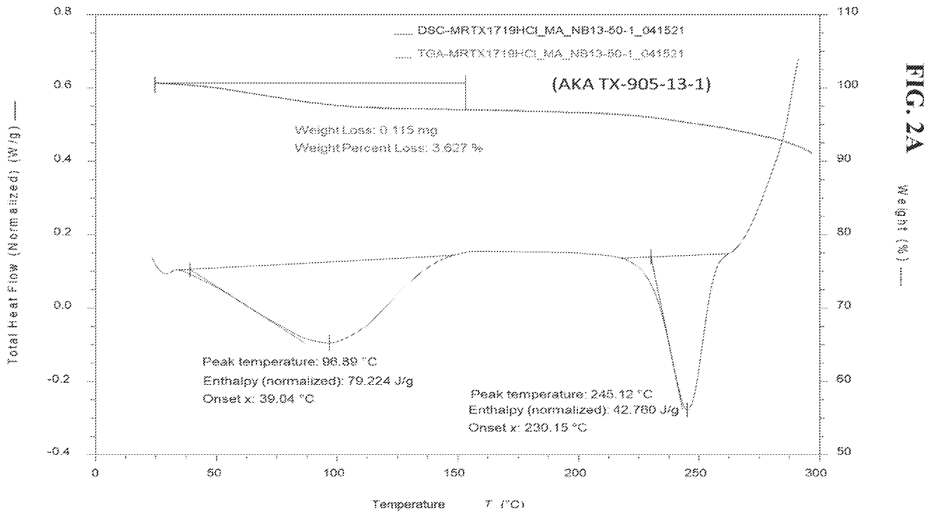

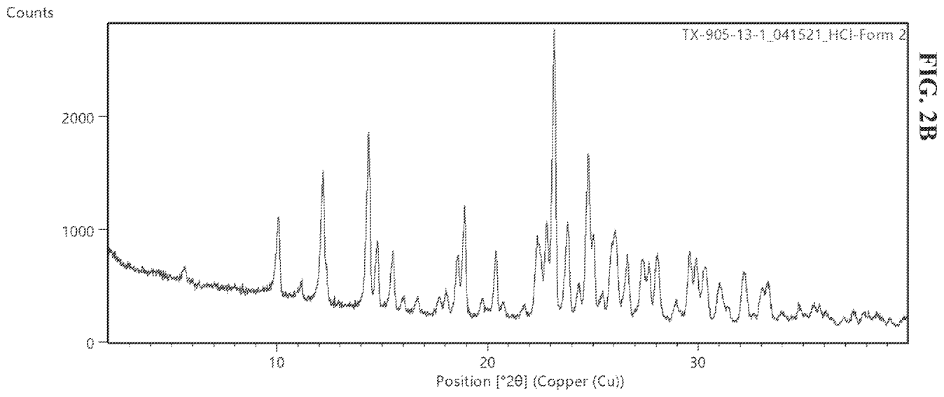

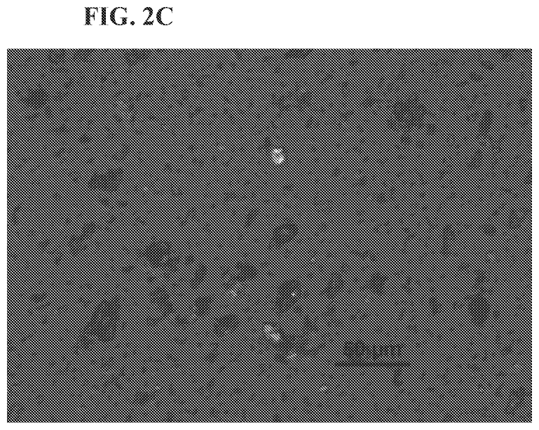

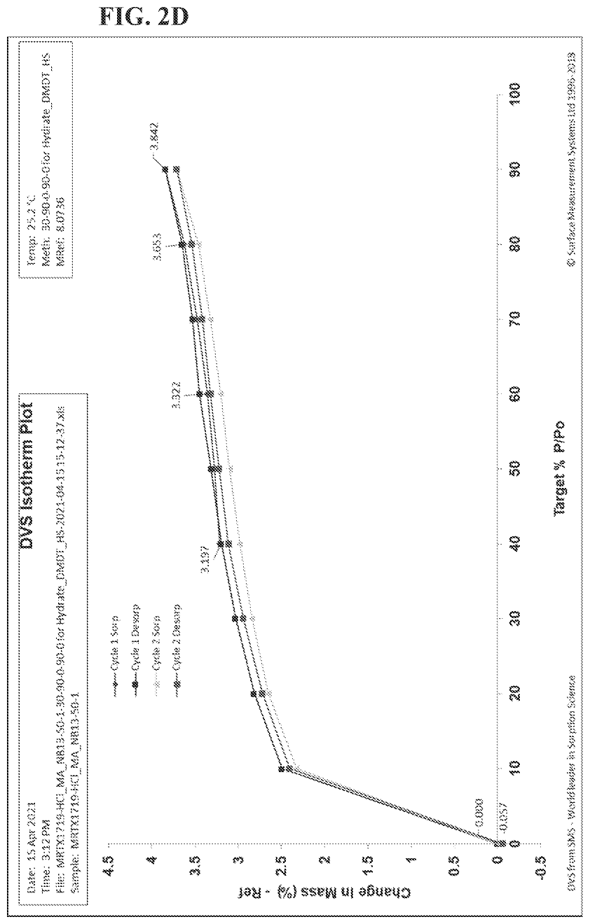

In one embodiment this disclosure provides a crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile hydrochloride salt, i.e., HCl salt Form A. In various embodiments, HCl salt Form A has a differential scanning calorimetric (DCS) thermogram (e.g., comprising endothermic peaks). In some embodiments, HCl salt Form A has an endothermic DSC peak temperature of about 118° C., e.g., within about ±2% of 118° C. In some embodiments, HCl salt Form A has an endothermic DSC peak temperature within ±1% of ±118° C. or within 0.5% of ±118° C. In some embodiments, HCl salt Form A has an endothermic DSC peak temperature of about 211° C., e.g., within about ±2% of 211° C. In some embodiments, HCl salt Form A has an endothermic DSC peak temperature within ±1% of 211° C. or within ±0.5% of 211° C. In various embodiments, HCl salt Form A has as a DSC thermogram with peak temperatures at about 118° C. (e.g., within about ±2% of 118° C., or ±1% of 118° C., or ±0.5% of 118° C.) and at about 211° C. (e.g., within about ±2% of 211° C., or ±1% of 211° C., or ±0.5% of 211° C.). For example, in some embodiments, HCl salt Form A has a DSC thermogram substantially shown in FIG. 2A.

In various embodiments, HCl salt Form A has an X-ray powder diffraction (XRPD) pattern. In some embodiments, HCl salt Form A has an XRPD pattern comprising a peak at a two-theta angle of 6.8°±0.2°. In some embodiments, HCl salt Form A has an XRPD pattern comprising a peak at a two-theta angle of 7.4°±0.2°. In some embodiments, HCl salt Form A has an XRPD pattern comprising a peak at a two-theta angle of 13.9°±0.2°. In some embodiments, HCl salt Form A has an XRPD pattern comprising a peak at a two-theta angle of 23.9°±0.2°. In some embodiments, HCl salt Form A has an XRPD pattern comprising a peak at a two-theta angle of 26.2°±0.2°. In some embodiments, HCl salt Form A has an XRPD pattern comprising peaks at a two-theta angles of 6.8°±0.2°, 7.4°±0.2°, 13.9°±0.2°, 23.9°±0.2°, and 26.2°±0.2°. For example, in some embodiments, HCl salt Form A has an XRPD pattern substantially shown in FIG. 2B. In some embodiments as described herein, HCl salt Form A is a trihydrate.

In some embodiments as described herein, HCl salt Form A has a thermal gravimetric analysis (TGA) plot comprising a mass loss of about 9.7% when heated from about 25° C. to about 150° C. For example, in some embodiments, HCl salt Form A has a TGA plot substantially shown in FIG. 2A.

In some embodiments as described herein, HCl salt Form A has a purity of at least 97% by weight of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile hydrochloride salt. In some embodiments, HCl salt Form A has a purity of at least 98% by weight of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile hydrochloride salt.

### HCl Salt Form B

Another embodiment this disclosure as described herein provides a crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile hydrochloride salt, i.e., HCl salt Form B. In various embodiments, HCl salt Form B has a differential scanning calorimetric (DCS) thermogram (e.g., comprising endothermic peaks). In some embodiments, HCl salt Form B has an endothermic DSC peak temperature of about 97° C., e.g., within about ±2% of 97° C. In some embodiments, HCl salt Form B has an endothermic DSC peak temperature within ±1% of 97° C. or within ±0.5% of 97° C. In some embodiments, HCl salt Form B has an endothermic DSC peak temperature of about 245° C., e.g., within about ±2% of 245° C. In some embodiments, HCl salt Form B has an endothermic DSC peak temperature within ±1% of 245° C. or within ±0.5% of 245° C. In various embodiments, HCl salt Form B has as a DSC thermogram with peak temperatures at about 97° C. (e.g., within about ±2% of 97° C., or 1% of 97° C., or 0.5% of 97° C.) and at about 245° C. (e.g., within about 2% of 245° C., or 1% of 245° C., or ±0.5% of 245° C.). For example, in some embodiments, HCl salt Form B has a DSC thermogram substantially shown in FIG. 2A.

In various embodiments, HCl salt Form B has an X-ray powder diffraction (XRPD) pattern. In some embodiments, HCl salt Form B has an XRPD pattern comprising a peak at a two-theta angle of 12.2°±0.2°. In some embodiments, HCl salt Form B has an XRPD pattern comprising a peak at a two-theta angle of 14.3±0.2°. In some embodiments, HCl salt Form B has an XRPD pattern comprising a peak at a two-theta angle of 18.9°±0.2°. In some embodiments, HCl salt Form B has an XRPD pattern comprising a peak at a two-theta angle of 23.2°±0.2°. In some embodiments, HCl salt Form B has an XRPD pattern comprising a peak at a two-theta angle of 26.0°±0.2°. In some embodiments, HCl salt Form B has an XRPD pattern comprising peaks at a two-theta angles of 12.2°±0.2°, 14.3 0.2°, 18.9°±0.2°, 23.2°±0.2°, and 26.0°±0.2°. For example, in some embodiments, HCl salt Form B has an XRPD pattern substantially shown in FIG. 2B. In some embodiments as described herein, HCl salt Form B is a mono-hydrate.

In some embodiments as described herein, HCl salt Form B has a thermal gravimetric analysis (TGA) plot comprising a mass loss of about 3.6% when heated from about 25° C. to about 150° C. For example, in some embodiments, HCl salt Form B has a TGA plot substantially shown in FIG. 2A.

In some embodiments as described herein, HCl salt Form B has a purity of at least 97% by weight of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile hydrochloride salt. In some embodiments, HCl salt Form B has a purity of at least 98% by weight of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile hydrochloride salt.

### HCl Salt Form E

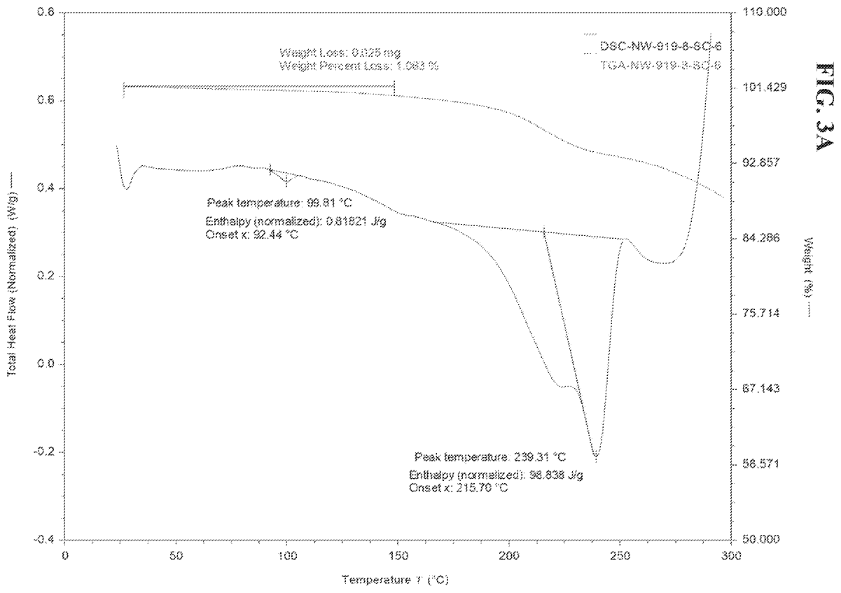

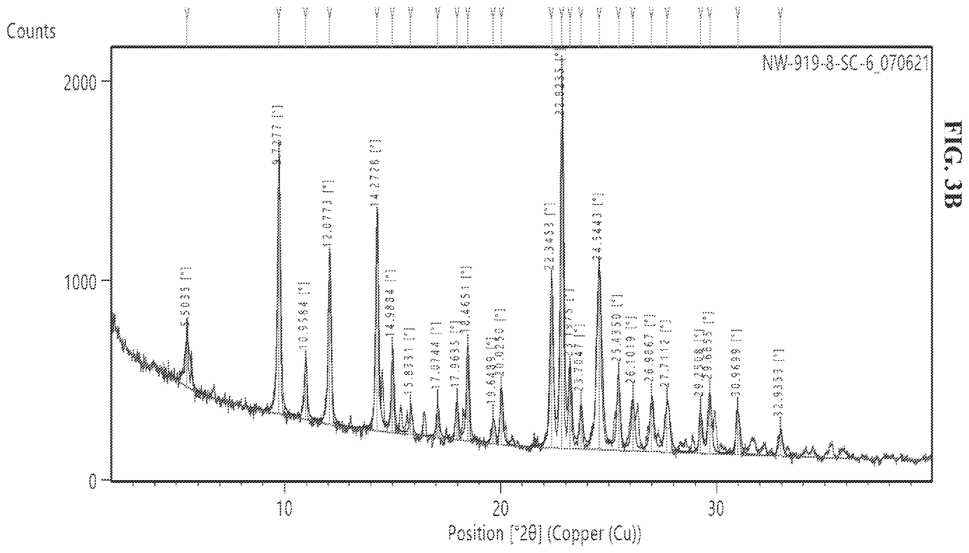

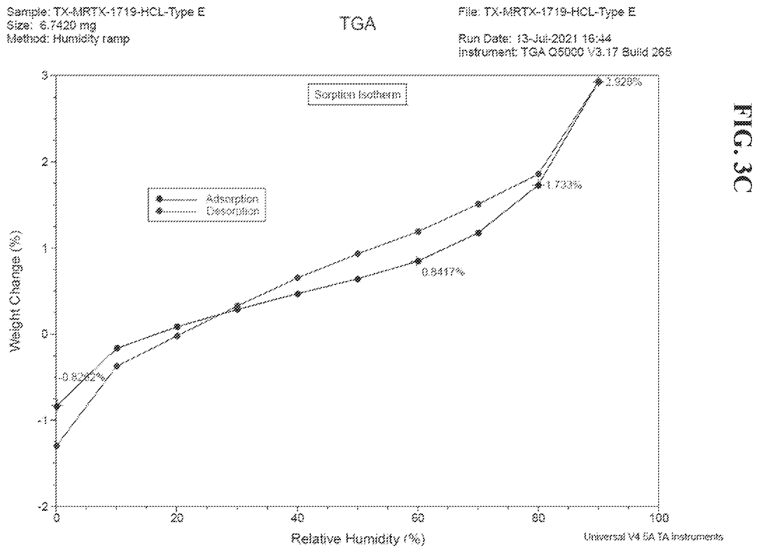

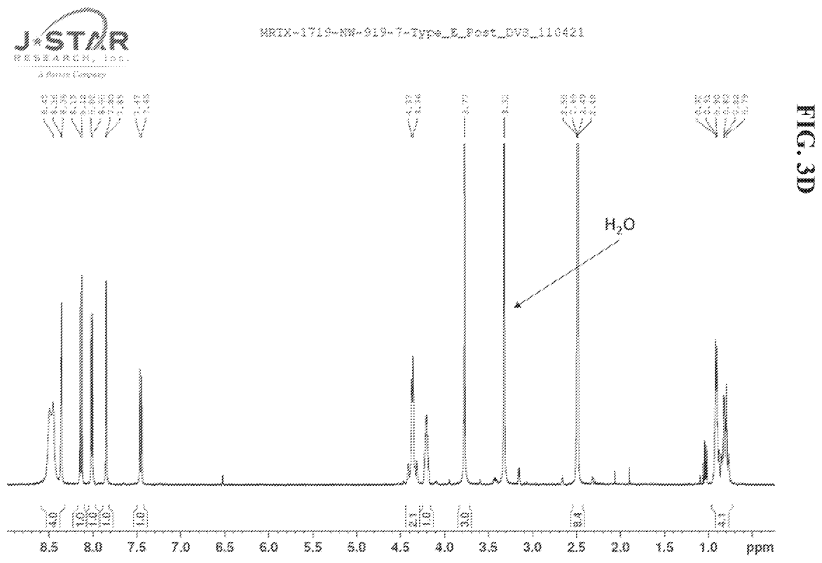

Another embodiment this disclosure as described herein provides a crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile hydrochloride salte, i.e., HCl salt Form E. In various embodiments, HCl salt Form E has a differential scanning calorimetric (DCS) thermogram (e.g., comprising endothermic peaks). In some embodiments, HCl salt Form E has an endothermic DSC peak temperature of about 239° C., e.g., within about ±2% of 239° C. In some embodiments, HCl salt Form E has an endothermic DSC peak temperature within ±1% of 239° C. or within ±0.5% of 239° C. For example, in some embodiments, HCl salt Form E has a DSC thermogram substantially shown in FIG. 3A.

In various embodiments, HCl salt Form E has an X-ray powder diffraction (XRPD) pattern. In some embodiments, HCl salt Form E has an XRPD pattern comprising a peak at a two-theta angle of 9.7°±0.2°. In some embodiments, HCl salt Form E has an XRPD pattern comprising a peak at a two-theta angle of 12.1°±0.2°. In some embodiments, HCl salt Form E has an XRPD pattern comprising a peak at a two-theta angle of 14.3°±0.2°. In some embodiments, HCl salt Form E has an XRPD pattern comprising a peak at a two-theta angle of 22.3°±0.2°. In some embodiments, HCl salt Form E has an XRPD pattern comprising a peak at a two-theta angle of 22.8°±0.2°. In some embodiments, HCl salt Form E has an XRPD pattern comprising a peak at a two-theta angle of 24.5°±0.2°. In some embodiments, HCl salt Form E has an XRPD pattern comprising peaks at a two-theta angles of 9.7°±0.2°, 12.1°±0.2°, 14.3°±0.2°, 22.3°±0.2°, 22.8°±0.2°, and 24.5°±0.2°. For example, in some embodiments, HCl salt Form E has an XRPD pattern substantially shown in FIG. 3B. In some embodiments as described herein, HCl salt Form E is an anhydrate.

In some embodiments as described herein, HCl salt Form E has a thermal gravimetric analysis (TGA) plot comprising a mass loss of about 1.0% when heated from about 25° C. to about 150° C. For example, in some embodiments, HCl salt Form E has a TGA plot substantially shown in FIG. 3A.

In some embodiments as described herein, HCl salt Form E has a purity of at least 97% by weight of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile hydrochloride salt. In some embodiments, HCl salt Form E has a purity of at least 98% by weight of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile hydrochloride salt.

### HCl Salt Form F

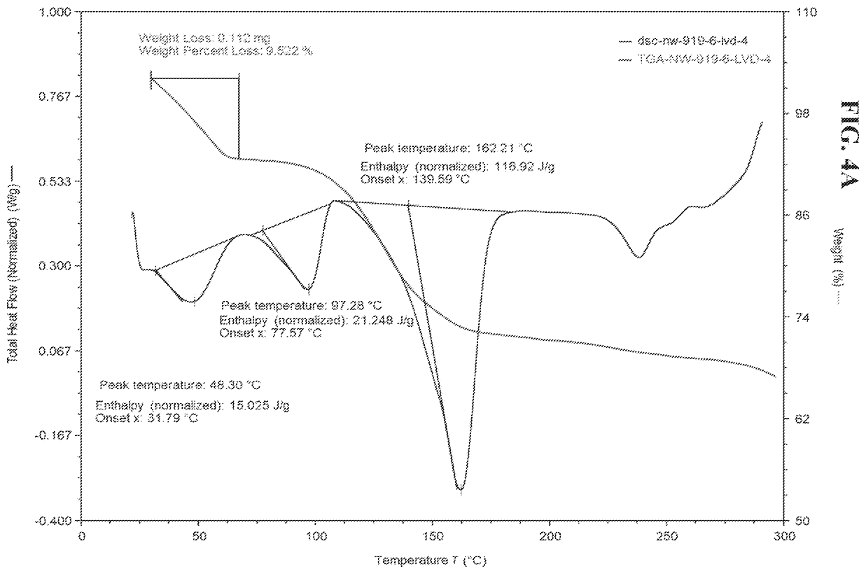

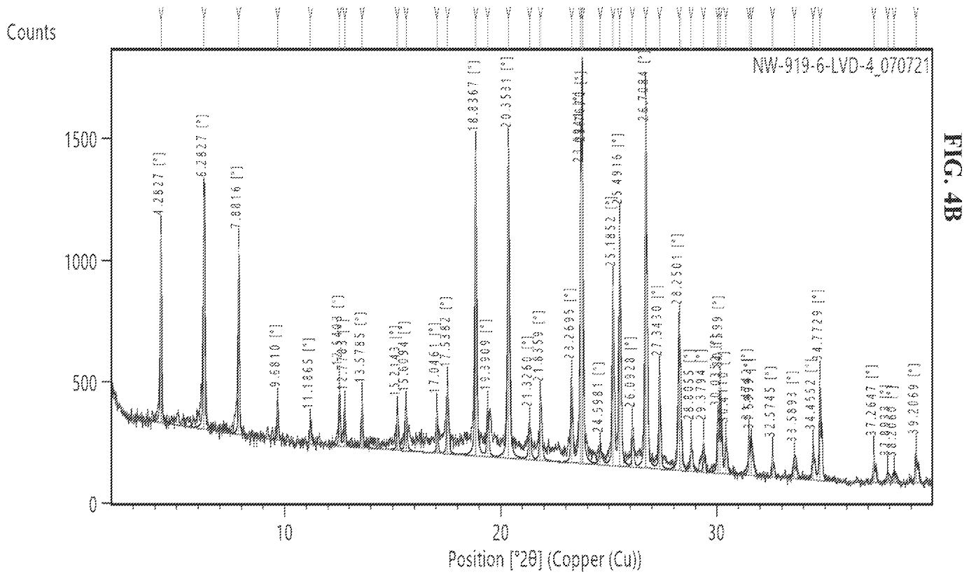

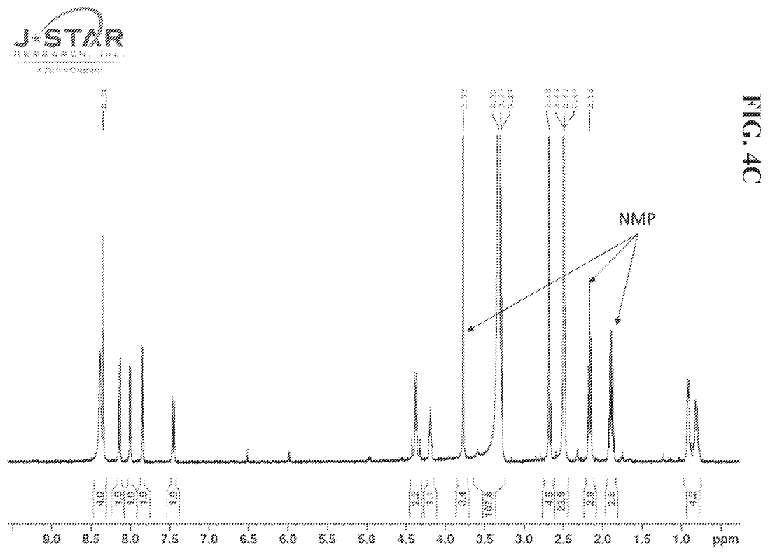

Another embodiment this disclosure as described herein provides a crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile hydrochloride salte, i.e., HCl salt Form F. In various embodiments, HCl salt Form F has a differential scanning calorimetric (DCS) thermogram (e.g., comprising endothermic peaks). In some embodiments, HCl salt Form F has an endothermic DSC peak temperature of about 15° C., e.g., within about ±2% of 15° C. In some embodiments, HCl salt Form F has an endothermic DSC peak temperature within ±1% of 15° C. or within ±0.5% of 15° C. In some embodiments, HCl salt Form F has an endothermic DSC peak temperature of about 97° C., e.g., within about 2% of 97° C. In some embodiments, HCl salt Form F has an endothermic DSC peak temperature within ±1% of 97° C. or within ±0.5% of 97° C. In some embodiments, HCl salt Form F has an endothermic DSC peak temperature of about 162° C., e.g., within about ±2% of 162° C. In some embodiments, HCl salt Form F has an endothermic DSC peak temperature within ±1% of 162° C. or within ±0.5% of 162° C. In various embodiments, HCl salt Form F has as a DSC thermogram with peak temperatures at about 15° C. (e.g., within about ±2% of 15° C., or 1% of 15° C., or ±0.5% of 15° C.), at about 97° C. (e.g., within about 2% of 97° C., or 1% of 97° C., or ±0.5% of 97° C.), and at about 162° C. (e.g., within about 2% of 162° C., or 1% of 162° C., or 0.5% of 162° C.). For example, in some embodiments, HCl salt Form F has a DSC thermogram substantially shown in FIG. 4A.

In various embodiments, HCl salt Form F has an X-ray powder diffraction (XRPD) pattern. In some embodiments, HCl salt Form F has an XRPD pattern comprising a peak at a two-theta angle of 4.3°±0.2°. In some embodiments, HCl salt Form F has an XRPD pattern comprising a peak at a two-theta angle of 6.3°±0.2°. In some embodiments, HCl salt Form F has an XRPD pattern comprising a peak at a two-theta angle of 7.9°±0.2°. In some embodiments, HCl salt Form F has an XRPD pattern comprising a peak at a two-theta angle of 18.8°±0.2°. In some embodiments, HCl salt Form F has an XRPD pattern comprising a peak at a two-theta angle of 20.3°±0.2°. In some embodiments, HCl salt Form F has an XRPD pattern comprising a peak at a two-theta angle of 23.7°±0.2°. In some embodiments, HCl salt Form F has an XRPD pattern comprising a peak at a two-theta angle of 26.7°±0.2°. In some embodiments, HCl salt Form F has an XRPD pattern comprising peaks at a two-theta angles of 4.3±0.2°, 6.3±0.2°, 7.9±0.2°, 18.8±0.2°, 20.3±0.2°, 23.7±0.2°, and 26.7±0.2°. For example, in some embodiments, HCl salt Form F has an XRPD pattern substantially shown in FIG. 4B. In some embodiments as described herein, HCl salt Form F is an n-methyl-2-pyrrolidone (NMP) solvate.

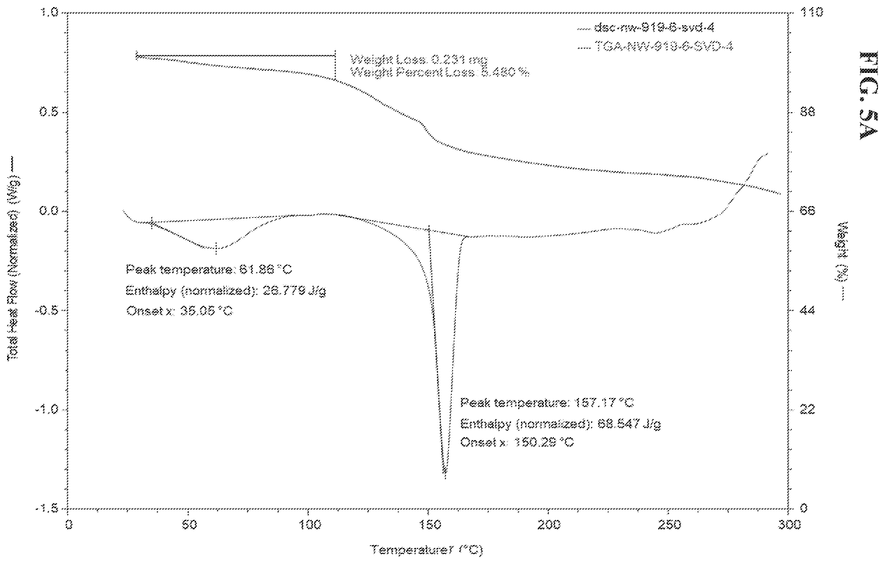

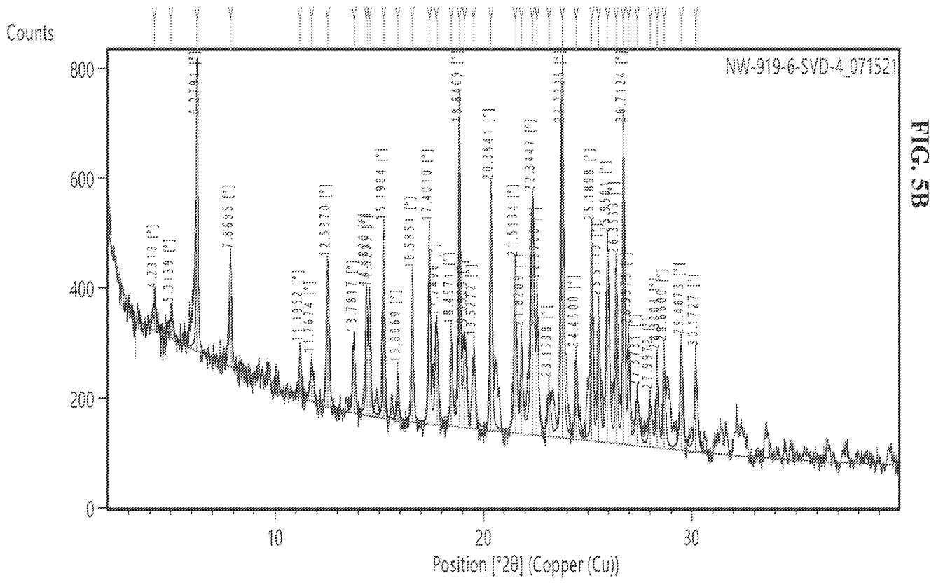

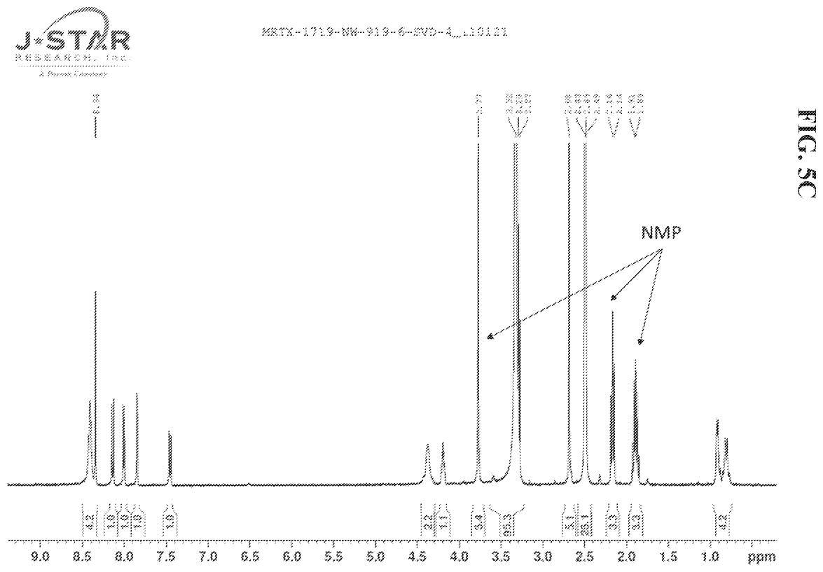

In some embodiments as described herein, HCl salt Form F has a thermal gravimetric analysis (TGA) plot comprising a mass loss of about 9.5% when heated from about 25° C. to about 150° C. For example, in some embodiments, HCl salt Form F has a TGA plot substantially shown in FIG. 5B4A

In some embodiments as described herein, HCl salt Form F has a purity of at least 97% by weight of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile hydrochloride salt. In some embodiments, HCl salt Form F has a purity of at least 98% by weight of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile hydrochloride salt.

### HCl Salt Form G

Another embodiment this disclosure as described herein provides a crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile hydrochloride salte, i.e., HCl salt Form G. In various embodiments, HCl salt Form G has a differential scanning calorimetric (DCS) thermogram (e.g., comprising endothermic peaks). In some embodiments, HCl salt Form G has an endothermic DSC peak temperature of about 62° C., e.g., within about ±2% of 62° C. In some embodiments, HCl salt Form G has an endothermic DSC peak temperature within ±1% of 62° C. or within ±0.5% of 62° C. For example, in some embodiments, HCl salt Form F has a DSC thermogram substantially shown in FIG. 5A.

In various embodiments, HCl salt Form G has an X-ray powder diffraction (XRPD) pattern. In some embodiments, HCl salt Form G has an XRPD pattern comprising a peak at a two-theta angle of 6.3°±0.2°. In some embodiments, HCl salt Form G has an XRPD pattern comprising a peak at a two-theta angle of 18.8°±0.2°. In some embodiments, HCl salt Form G has an XRPD pattern comprising a peak at a two-theta angle of 20.3°±0.2°. In some embodiments, HCl salt Form G has an XRPD pattern comprising a peak at a two-theta angle of 22.3°±0.2°. In some embodiments, HCl salt Form G has an XRPD pattern comprising a peak at a two-theta angle of 23.8°±0.2°. In some embodiments, HCl salt Form G has an XRPD pattern comprising a peak at a two-theta angle of 26.7°±0.2°. In some embodiments, HCl salt Form F has an XRPD pattern comprising peaks at a two-theta angles of 6.3°±0.2°, 18.8°±0.2°, 20.3°±0.2°, 22.3°±0.2°, 23.8°±0.2°, and 26.7°±0.2°. For example, in some embodiments, HCl salt Form G has an XRPD pattern substantially shown in FIG. 5B. In some embodiments as described herein, HCl salt Form G is an n-methyl-2-pyrrolidone (NMP) solvate.

In some embodiments as described herein, HCl salt Form G has a thermal gravimetric analysis (TGA) plot comprising a mass loss of about 5.5% when heated from about 25° C. to about 150° C. For example, in some embodiments, HCl salt Form G has a TGA plot substantially shown in FIG. 5A.

In some embodiments as described herein, HCl salt Form G has a purity of at least 97% by weight of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile hydrochloride salt. In some embodiments, HCl salt Form G has a purity of at least 98% by weight of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile hydrochloride salt.

### Processes of Preparing Crystalline Forms of HCl Salts of Compound 1

In another aspect, this disclosure provides processes of preparing crystalline forms of HCl salt of Compound 1, e.g., HCl salt Form A, HCl salt Form B, HCl salt Form E, HCl salt Form F, and HCl salt Form G. Crystalline forms of HCl salt Form A, HCl salt Form B, HCl salt Form E, HCl salt Form F, and HCl salt Form G can be made by a variety of methods as discussed in the Examples below. For example, the crystalline forms as described herein may be prepared by slurry methods, anti-solvent addition, solid vapor diffusion, liquid vapor diffusion, slow evaporation at room temperature, slow cooling, or polymer induced crystallization.

In various embodiments, the slurry method can be conducted at a variety of temperatures and with a variety of solvents. For example, in some embodiments, the slurry method is conducted at room temperature or at an elevated temperature (e.g., 50° C.). To prepare crystalline forms with the slurry method, amorphous HCl salt of Compound 1 is suspended and stirred in a solvent at a temperature (e.g., room temperature or elevated temperature) to provide solids.

In various embodiments, the anti-solvent addition method can be conducted at a variety of temperatures and with a variety of solvents. For example, in some embodiments, the anti-solvent addition method is conducted at room temperature. To prepare crystalline forms with the anti-solvent addition method, amorphous HCl salt of Compound 1 is dissolved in a solvent to obtain a saturated solution and an anti-solvent is added in an amount of up to, for example, 20 times in volume to provide solids.

In various embodiments, the solid vapor diffusion method can be conducted at a variety of temperatures and with a variety of solvents. For example, in some embodiments, the solid vapor diffusion method is conducted at room temperature. To prepare crystalline forms with the solid vapor diffusion method, amorphous HCl salt of Compound 1 is provided in a first vial which is placed in a second vial containing a solvent. There is not physical contact between solid free base of Compound 1 in the first vial and the solvent in the second vial. The solids are then characterized after 14 days.

In various embodiments, the liquid vapor diffusion method can be conducted at a variety of temperatures and with a variety of solvents. For example, in some embodiments, the liquid vapor diffusion method is conducted at room temperature. To prepare crystalline forms with the liquid vapor diffusion method, amorphous HCl salt of Compound 1 is dissolved in a solvent to obtain a saturated solution in a first vial and the first vial is then placed in a second vial containing an anti-solvent to provide solids.

In various embodiments, the slow evaporation method can be conducted at a variety of temperatures and with a variety of solvents. For example, in some embodiments, the slow evaporation method is conducted at room temperature or at elevated temperatures (e.g., 50° C.). To prepare crystalline forms with the slow evaporation method, amorphous HCl salt of Compound 1 is dissolved in a solvent to obtain a saturated solution. The vial is then covered with paraffin film with a plurality, e.g., 3-5, holes and allowed to evaporate to provide solids.

In various embodiments, the slow cooling method can be conducted at a variety of temperatures and with a variety of solvents. For example, in some embodiments, the slow cooling method is conducted at elevated temperatures (e.g., 55° C.). To prepare crystalline forms with the slow cooling method, amorphous HCl salt of Compound 1 is dissolved in a solvent to obtain a saturated solution, for example, in the range of 40-70° C., or 45-70° C., or 50-70° C., or 40-65° C., or 45-65° C., or 50-65° C., or 40-60° C., or 45-60° C., or 50-60° C. In some embodiments, to prepare the crystalline forms with the slow cooling method, free base of Compound 1 is dissolved in a solvent to obtain a saturated solution, for example, at approximately 55° C. The solution is slowly cooled down to room temperature to provide solids.

In various embodiments, the polymer induced crystallization method can be conducted at a variety of temperatures and with a variety of solvents. For example, in some embodiments, the polymer induced crystallization is conducted at room temperature. To prepare crystalline forms with the polymer induced crystallization method, amorphous HCl salt of Compound 1 is dissolved in a solvent to provide a saturated solution. A polymer is then added to the saturated solution to induce heteronucleation and provide solids.

In some embodiments, to provide HCl salt Form A, slurry methods or solid vapor diffusion methods may be used. When a slurry method is used, the solvent may be water. When solid vapor diffusion is used, the solvent may be water.

In some embodiments, to provide HCl salt Form B, slurry methods or solid vapor diffusion methods may be used. When a slurry method is used, the solvent may be selected from acetonitrile (ACN) or isopropyl alcohol/water. When solid vapor diffusion method is used, the solvent may be ACN.

In some embodiments, to provide HCl salt Form E, slurry methods, anti-solvent addition, solid vapor diffusion, liquid vapor diffusion, slow evaporation at room temperature, slow cooling, or polymer induced crystallization methods may be used. When a slurry method is used, the solvent may be selected from methanol (MeOH), n-propyl alcohol (NPA), dichloromethane (DCM), ethanol (EtOH), ACN, tetrahydrofuran (THF)/n-heptane, IPA/toluene, and MeOH/water. When anti-solvent addition is used, the solvent may be MeOH and the anti-solvent may be selected from methyl tert-butyl ether (MTBE), ACN, and heptane. When solid vapor diffusion is used, the solvent may be EtOH. When liquid vapor diffusion is used, the solvent may be MeOH and the anti-solvent may be selected from cyclopentyl methyl ether (CPME), ACN, and isopropyl acetate (IPAc). When slow evaporation is used, the solvent may be selected from MeOH, IPA, and 1-butanol (1-BuOH). When slow cooling method is used, the solvent may be selected from MeOH and EtOH. When polymer induced crystallization is used, the solvent may be MeOH and the polymer may be polyvinylpyrrolidone (PVP).

In some embodiments, to provide HCl salt Form F, liquid vapor diffusion may be used. When liquid vapor diffusion is used, the solvent may be n-methylpyrrolidone (NMP) and the anti-solvent may be selected from heptance or MTBE.

In some embodiments, to provide HCl salt Form F, solid vapor diffusion may be used. When liquid vapor diffusion is used, the solvent may be NMP.

### Pharmaceutical Compositions

In another aspect, this disclosure provides pharmaceutical comprisings a crystalline form of Compound 1 (e.g., HCl salt Form A, HCl salt Form B, HCl salt Form E, HCl salt Form F, and HCl salt Form G) and an appropriate carrier, excipient or diluent. The exact nature of the carrier, excipient or diluent will depend upon the desired use for the composition, and may range from being suitable or acceptable for veterinary uses to being suitable or acceptable for human use. The composition may optionally include one or more additional compounds. In certain embodiments, the composition may include one or more antibiotic compounds. In another aspect, this disclosure provides pharmaceutical compositions comprising HCl salt Form A and a pharmaceutically acceptable carrier. In another aspect, this disclosure provides pharmaceutical compositions comprising HCl salt Form B and a pharmaceutically acceptable carrier. In another aspect, this disclosure provides pharmaceutical compositions comprising HCl salt Form E and a pharmaceutically acceptable carrier. In another aspect, this disclosure provides pharmaceutical compositions comprising HCl salt Form F and a pharmaceutically acceptable carrier. In another aspect, this disclosure provides pharmaceutical compositions comprising HCl salt Form G and a pharmaceutically acceptable carrier.

When used to treat or prevent such diseases, Compound 1 described herein may be administered singly, as mixtures of one or more compounds or in mixture or combination with other agents useful for treating such diseases and/or the symptoms associated with such diseases. The compounds may also be administered in mixture or in combination with agents useful to treat other disorders or maladies, such as steroids, membrane stabilizers, 5LO inhibitors, leukotriene synthesis and receptor inhibitors, inhibitors of IgE isotype switching or IgE synthesis, IgG isotype switching or IgG synthesis, 0-agonists, tryptase inhibitors, aspirin, COX inhibitors, methotrexate, anti-TNF drugs, retuxin, PD4 inhibitors, p38 inhibitors, PDE4 inhibitors, and antihistamines, to name a few. The Compound 1 may be administered in the crystalline forms as described herein, or as pharmaceutical compositions comprising the crystalline forms as described herein.

Pharmaceutical compositions comprising the various crystalline forms of Compound 1 may be manufactured by means of conventional mixing, dissolving, granulating, dragee-making levigating, emulsifying, encapsulating, entrapping or lyophilization processes. The compositions may be formulated in conventional manner using one or more physiologically acceptable carriers, diluents, excipients or auxiliaries which facilitate processing of the compounds into preparations which can be used pharmaceutically.

Pharmaceutical compositions may take a form suitable for virtually any mode of administration, including, for example, topical, ocular, oral, buccal, systemic, nasal, injection, transdermal, rectal, vaginal, etc., or a form suitable for administration by inhalation or insufflation.

For topical administration, the compound(s) may be formulated as solutions, gels, ointments, creams, suspensions, etc. as are well-known in the art. Systemic formulations include those designed for administration by injection, e.g., subcutaneous, intravenous, intramuscular, intrathecal or intraperitoneal injection, as well as those designed for transdermal, transmucosal oral or pulmonary administration.

Useful injectable preparations include sterile suspensions, solutions or emulsions of the active compound(s) in aqueous or oily vehicles. The compositions may also contain formulating agents, such as suspending, stabilizing and/or dispersing agent. The formulations for injection may be presented in unit dosage form, e.g., in ampules or in multidose containers, and may contain added preservatives. Alternatively, the injectable formulation may be provided in powder form for reconstitution with a suitable vehicle, including but not limited to sterile pyrogen free water, buffer, dextrose solution, etc., before use. To this end, the active compound(s) may be dried by any art-known technique, such as lyophilization, and reconstituted prior to use.

For transmucosal administration, penetrants appropriate to the barrier to be permeated are used in the formulation. Such penetrants are known in the art.

For oral administration, the pharmaceutical compositions may take the form of, for example, lozenges, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art with, for example, sugars, films or enteric coatings.

Liquid preparations for oral administration may take the form of, for example, elixirs, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol, Cremophore™ or fractionated vegetable oils); and preservatives (e.g., methyl or propyl-p-hydroxybenzoates or sorbic acid). The preparations may also contain buffer salts, preservatives, flavoring, coloring and sweetening agents as appropriate.

Preparations for oral administration may be suitably formulated to give controlled release of the compound, as is well known. For buccal administration, the compositions may take the form of tablets or lozenges formulated in conventional manner. For rectal and vaginal routes of administration, the compound(s) may be formulated as solutions (for retention enemas) suppositories or ointments containing conventional suppository bases such as cocoa butter or other glycerides.

For nasal administration or administration by inhalation or insufflation, the compound(s) can be conveniently delivered in the form of an aerosol spray from pressurized packs or a nebulizer with the use of a suitable propellant, e.g., dichlorodifluoromethane, trichlorofluoromethane, dichlorotetrafluoroethane, fluorocarbons, carbon dioxide or other suitable gas. In the case of a pressurized aerosol, the dosage unit may be determined by providing a valve to deliver a metered amount. Capsules and cartridges for use in an inhaler or insufflator (for example capsules and cartridges comprised of gelatin) may be formulated containing a powder mix of the compound and a suitable powder base such as lactose or starch.

For ocular administration, the compound(s) may be formulated as a solution, emulsion, suspension, etc. suitable for administration to the eye. A variety of vehicles suitable for administering compounds to the eye are known in the art.

For prolonged delivery, the compound(s) can be formulated as a depot preparation for administration by implantation or intramuscular injection. The compound(s) may be formulated with suitable polymeric or hydrophobic materials (e.g., as an emulsion in an acceptable oil) or ion exchange resins, or as sparingly soluble derivatives, e.g., as a sparingly soluble salt. Alternatively, transdermal delivery systems manufactured as an adhesive disc or patch which slowly releases the compound(s) for percutaneous absorption may be used. To this end, permeation enhancers may be used to facilitate transdermal penetration of the compound(s).

Alternatively, other pharmaceutical delivery systems may be employed. Liposomes and emulsions are well-known examples of delivery vehicles that may be used to deliver compound(s). Certain organic solvents such as dimethyl sulfoxide (DMSO) may also be employed, although usually at the cost of greater toxicity.

The pharmaceutical compositions may, if desired, be presented in a pack or dispenser device which may contain one or more unit dosage forms containing the compound(s). The pack may, for example, comprise metal or plastic foil, such as a blister pack. The pack or dispenser device may be accompanied by instructions for administration.

### Methods of Use

The crystalline forms described herein, or compositions thereof, will generally be used in an amount effective to achieve the intended result, for example in an amount effective to treat or prevent the particular disease being treated. By therapeutic benefit is meant eradication or amelioration of the underlying disorder being treated and/or eradication or amelioration of one or more of the symptoms associated with the underlying disorder such that the patient reports an improvement in feeling or condition, notwithstanding that the patient may still be afflicted with the underlying disorder. Therapeutic benefit also generally includes halting or slowing the progression of the disease, regardless of whether improvement is realized.

In yet another aspect, the invention provides for methods for inhibiting PRMT5 activity in a cell, comprising contacting the cell in which inhibition of PRMT5 activity is desired in vitro with an effective amount of a crystalline forms of Compound 1 as described herein or pharmaceutical compositions containing the crystalline forms of Compound 1 as described herein. In one embodiment, the cell is an MTAP-deficient cell.

The compositions and methods provided herein are particularly deemed useful for inhibiting PRMT5 activity in a cell in vivo. In one embodiment, a cell in which inhibition of PRMT5 activity is desired is contacted in vivo with a therapeutically effective amount of crystalline forms of Compound 1 as described herein or pharmaceutical compositions containing the crystalline forms of Compound 1 as described herein. In one embodiment, the cell is an MTAP-deficient cell. In one embodiment, the negatively modulating the activity of PRMT5 occurs in the presence of bound MTA.

By negatively modulating the activity of PRMT5, particularly in cases for cells that lack MTAP activity, the methods are designed to inhibit PRMT5 activity to block cellular proliferation. The cells may be contacted in a single dose or multiple doses in accordance with a particular treatment regimen to affect the desired negative modulation of PRMT5. The degree PRMT5 inhibition may be monitored in vitro against the enzyme in the presence and absence of MTA and in the cell using well known methods, including those described in Example B below, to assess the effectiveness of treatment and dosages.

In another aspect, methods of treating cancer comprising administering to a patient having cancer a therapeutically effective amount of crystalline forms of Compound 1 as described herein or pharmaceutical compositions containing the crystalline forms of Compound 1 as described herein. In one embodiment, the cancer is an MTAP-associated cancer.

The compositions and methods provided herein may be used for the treatment of a wide variety of cancer including tumors such as prostate, breast, brain, skin, cervical carcinomas, testicular carcinomas, etc. More particularly, cancers that may be treated by the compositions and methods of the invention include, but are not limited to tumor types such as astrocytic, breast, cervical, colorectal, endometrial, esophageal, gastric, head and neck, hepatocellular, laryngeal, lung, oral, ovarian, prostate and thyroid carcinomas and sarcomas. More specifically, these compounds can be used to treat: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Kaposi's sarcoma, moles dysplastic nevi, lipoma, angioma, dermatofibroma, keloids, psoriasis; and Adrenal glands: neuroblastoma. In certain embodiments, the cancer is diffuse large B-cell lymphoma (DLBCL).

In one embodiment, the cancer is an MTAP-associated cancer selected from hepatocellular carcinoma, breast cancer, skin cancer, bladder cancer, liver cancer, pancreatic cancer, and head and neck cancer.

In other embodiments, the cancer is selected from the group consisting of ovarian serous cystadenocarcinoma, squamous cell lung cancer, lung adenocarcinoma, mesothelioma; esophogeal squamous cell carcinoma, gastric adenocarcinoma, pancreatic ductal adenocarcinoma, kidney adenocarcinoma, bladder transitional cell carcinoma, hepatocellular carcinoma, cholangiocarcinoma, cholangiocarcinoma; osteosarcoma, multiple myeloma, astrocytoma, glioma, glioblastoma, uterine sarcoma, acute myeloid leukemia, acute lymphoblastic leukemia, non-Hodgkin's lymphoma, malignant melanoma, endometrial carcinoma and thyroid carcinoma.

In other embodiments, the cancer is selected from the group consisting of bladder cancer, bone cancer, brain cancer, blood cancer, breast cancer, colorectal cancer, esophageal cancer, gastric cancer, head and neck cancer, kidney cancer, liver cancer, lung cancer, mesothelioma, ovarian cancer, pancreatic cancer, skin cancer, thyroid cancer and uterine cancer.

The concentration and route of administration to the patient will vary depending on the cancer to be treated. The crystalline forms of Compound 1 as described herein or pharmaceutical compositions containing the crystalline forms of Compound 1 as described herein also may be co-administered with other anti-neoplastic compounds, e.g., chemotherapy, or used in combination with other treatments, such as radiation or surgical intervention, either as an adjuvant prior to surgery or post-operatively.

### EXAMPLES

The following Examples are intended to illustrate further certain embodiments of the invention and are not intended to limit the scope of the invention.

### Example 1: Characterizing of Amorphous HCl Salt of Compound 1

Compound 1 can be prepared as a gum according to procedures disclosed in published International Application No. WO2021050915. See Example 16-8.

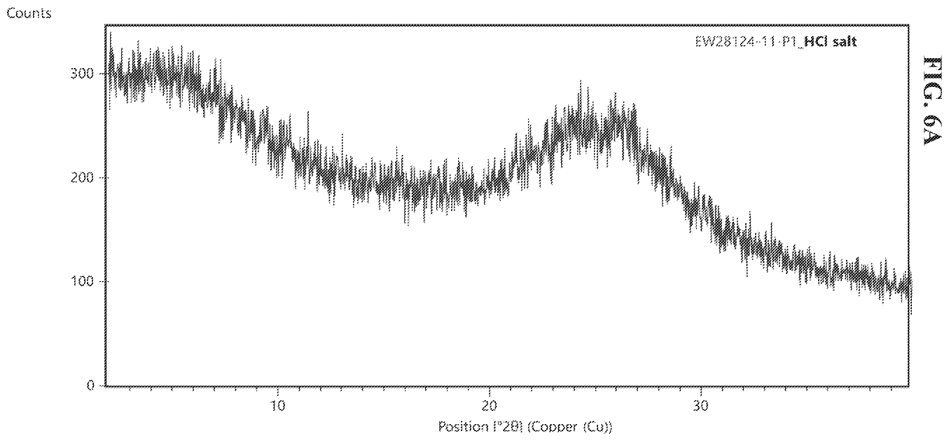

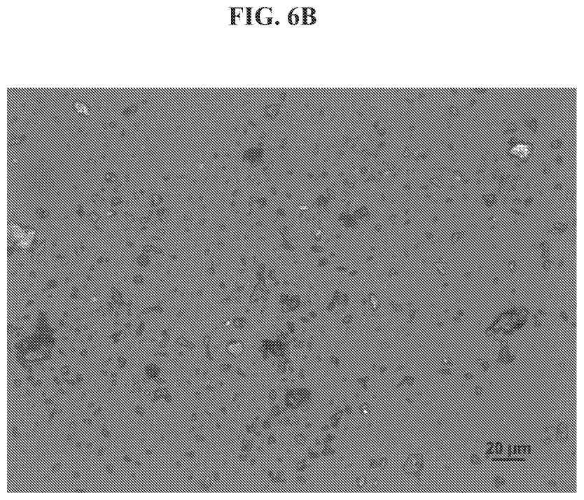

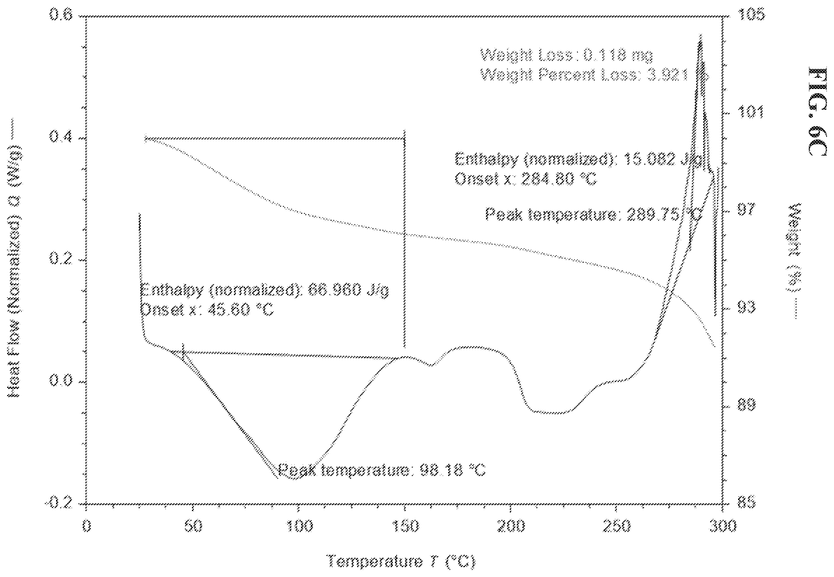

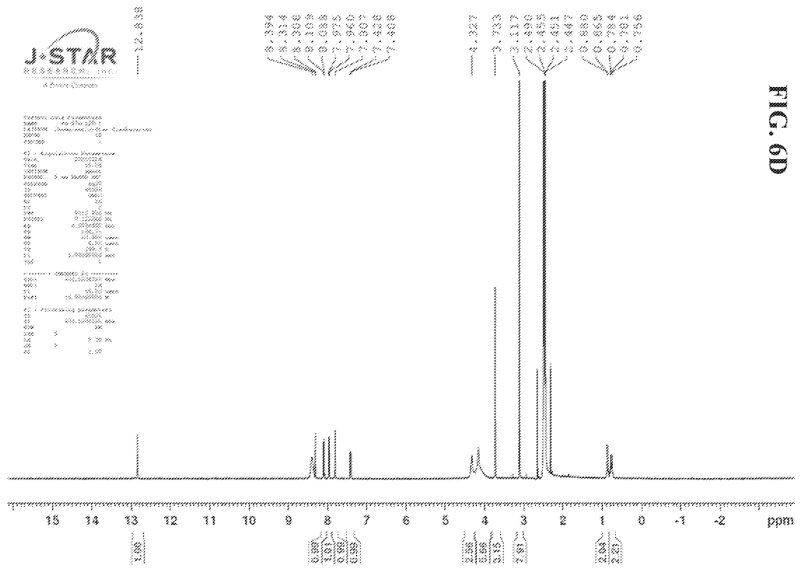

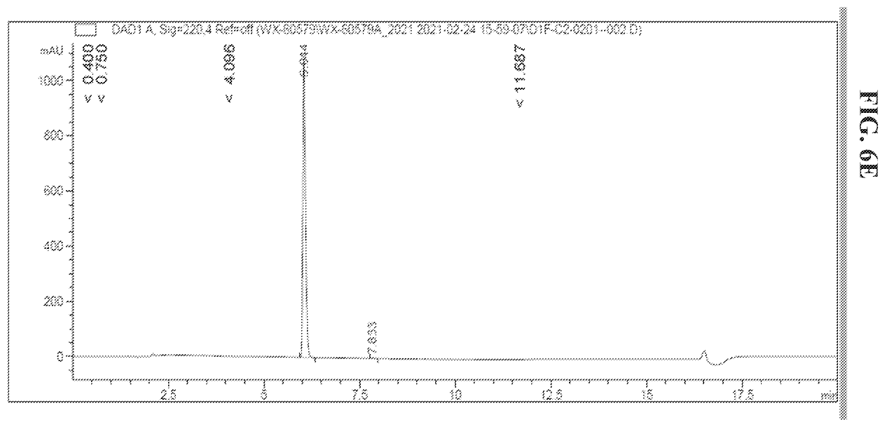

An amorphous salt of Compound 1 (2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile), provided as white solids, was characterized by XRPD, PLM, DSC, TGA, ¹H-NMR, and HPLC. The results are summarized in Table 1 and shown in FIGS. 6A-6E.

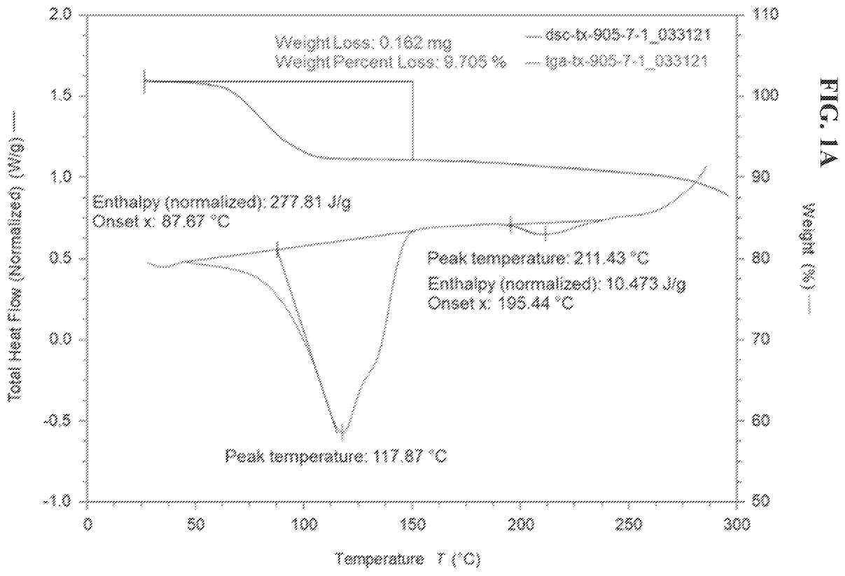

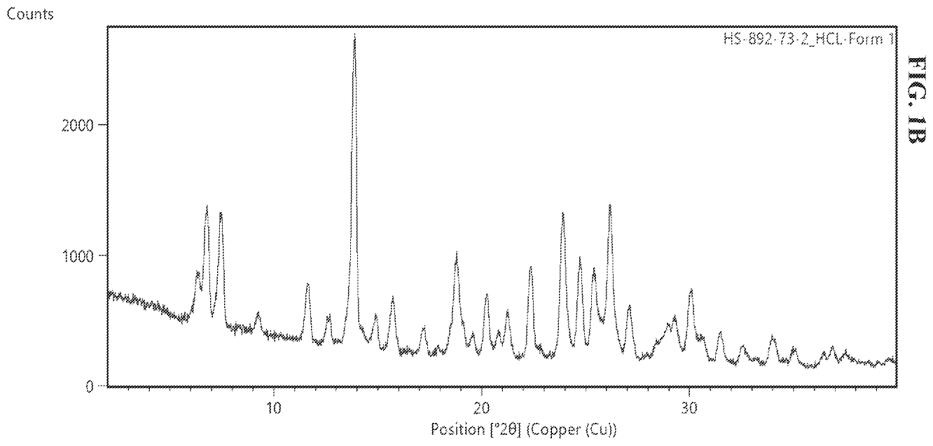

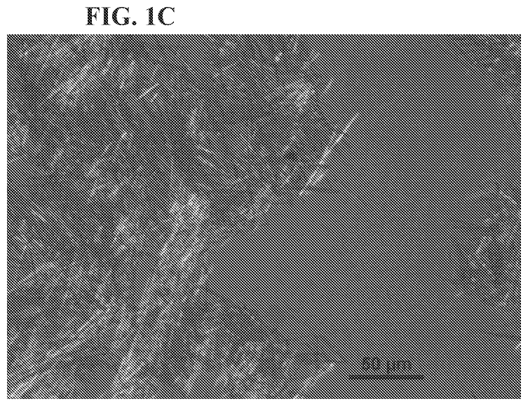

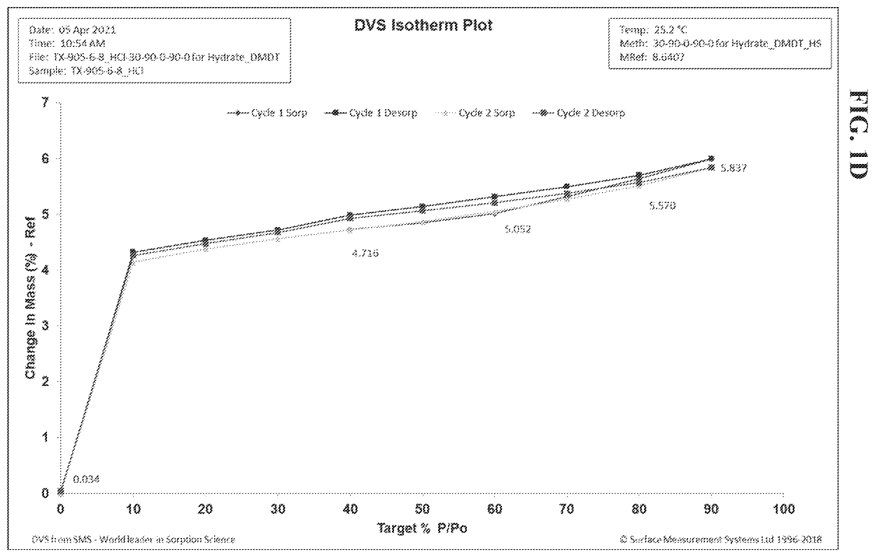

TABLE 1

| Technique | Data | Results |
|---|---|---|
| XRPD | FIG. 1A | Amorphous |
| PLM | FIG. 1B | Irregular shape particles |
| DSC/TGA | FIG. 1C | Endotherm at 98.2° C. (peak temp) |
| | | 3.9% weight loss observed before 150° C. |
| NMR | FIG. 1D | Consistent with chemical structure |
| HPLC | FIG. 1E | 99.9 A % |

### Example 2: Formation of HCl Form a of Compound 1

The amorphous HCl salt of Compound 1 (30 mg) was suspended in 2 mL of water at room temperature. The solids in the slurry were then characterized after 14 days by XRPD, PLM, DSC, TGA, and DVS. The crystals produced were an off-white solid. The results of the characterization of summarized in Table 2 and shown in FIGS. 1A-ID.

TABLE 2

| Sample | HCl Form A |
|---|---|
| Crystallinity by XRPD | High |
| Wt. Loss in TGA (%, ° C.) | 9.71 (150) |
| Endotherm in DSC (peak, ° C.) | 117.87 |
| | 211.43 |
| Heat of fusion (J/g) | 277.81 |
| | 10.47 |
| Hygroscopiscity (25° C./80% RH) | 1.43% (slightly |
| | hygroscopic) |
| Form | Tri-hydrate |

Other process for preparing crystalline forms of Compound 1 where also evaluated to provide HCl salt Form A. Four additional methods were found to provide HCl salt Form A: slurry at 50° C., solid vapor diffusion, slow evaporation, and slow cooling.

For the slurry experiments at 50° C., approximately 30 mg of amorphous HCl salt of Compound 1 was suspended and stirred in 2 mL of water in a 4 mL vial at 50° C. The solids in the slurry were characterized by XRPD after 6 days and identified as HCl Form A.

For the solid vapor diffusion experiments, approximately 30 mg of amorphous HCl salt of Compound 1 was kept in a 4 mL vial which were placed in 20 mL glass vial containing water. The solids were characterized by XRPD after 14 days and identified as HCl Form A.

For the slow evaporation experiments, evaporation occurred at room temperature. To obtain a saturated solution, approximately 30 mg of amorphous HCl salt of Compound 1 was dissolved in water in a 4 mL vial. The vial was covered with paraffin film with 3-5 holes and placed at room temperature for evaporation. The obtained solids were characterized by XRPD and identified as HCl Form A.

For the slow cooling experiments, approximately 30 mg of amorphous HCl salt of Compound 1 was dissolved in water to a obtain saturated solution in a 4 mL vial at 55° C. using a hot plate. The solution was slowly cooled down to room temperature using the following ramp: 55° C. for 3 hours, 40° C. for 4 hours, 25° C. for 4 hours, 15° C. for 5 hours, and 10° C. for 5 hours. The obtained solids were characterized by XRPD and identified as HCl salt Form A.

Table 3 describes the XRD pattern of HCl Form A crystalline form of Compound 1 shown in FIG. 1B.

TABLE 3

| Pos. (°2θ) | Intensity | Height (cts) |
|---|---|---|
| 6.3135 | Low | 382.58 |
| 6.7826 | Medium | 909.73 |
| 7.4184 | Medium | 892.96 |
| 9.2422 | Low | 132.69 |
| 11.6321 | Low | 438.54 |
| 12.7077 | Low | 187.92 |
| 13.8838 | High | 2422.8 |
| 14.9149 | Low | 241.42 |
| 15.7133 | Low | 393.08 |
| 17.2135 | Low | 177.61 |
| 17.8945 | Low | 47.65 |
| 18.7799 | Medium | 746.08 |
| 19.5942 | Low | 157.83 |
| 20.2563 | Low | 475.1 |
| 20.816 | Low | 185.28 |
| 21.2442 | Low | 336.76 |
| 22.3713 | Medium | 701.52 |
| 23.8295 | Medium | 911.61 |
| 23.9138 | Medium | 1087.91 |
| 24.7333 | Medium | 780.7 |
| 25.404 | Medium | 690.08 |
| 26.1724 | Medium | 1214.32 |
| 27.0933 | Low | 407.48 |
| 28.3499 | Low | 135.19 |
| 28.9734 | Low | 272.01 |
| 29.2718 | Low | 337.29 |
| 30.0976 | Low | 546.63 |
| 30.5954 | Low | 189.92 |
| 31.4675 | Low | 233.42 |
| 32.5444 | Low | 136.7 |
| 33.9823 | Low | 220.76 |
| 34.9735 | Low | 127.27 |
| 36.4438 | Low | 96.52 |
| 36.8753 | Low | 139.61 |
| 37.4832 | Low | 95.36 |

The hygroscopicity of HCl Form A was also evaluated by conducting dynamic vapor sorption experiments. FIG. 1D shows the DVS isotherm of HCl Form A which shows a water uptake of approximately 1.43% at 25° C. between 10% relative humidity and 90% relative humidity. No form change was observed for the sample after DVS evaluation.

### Example 3: Formation of HCl Form B of Compound 1

The amorphous HCl salt of Compound 1 (30 mg) was suspended in 2 mL of acetonitrile at room temperature. The solids in the slurry were there then characterized after 14 days by XRPD, PLM, DSC, TGA, and DVS. The crystals produced were an off-white solid. The results of the characterization of summarized in Table 4 and shown in FIGS. 2A-2D.

TABLE 4

| Sample | HCl Form B |
|---|---|
| Crystallinity by XRPD | High |
| Wt. Loss in TGA (%, ° C.) | 3.63 (150) |
| Endotherm in DSC (peak, ° C.) | 96.89 |
| | 245.12 |
| Heat of fusion (J/g) | 79.22 |
| | 42.76 |
| Hygroscopicity (25° C./80% RH) | 1.3% (slightly |
| | hygroscopic) |
| Form | Mono-hydrate |

Other processes for preparing crystalline forms of Compound 1 where also evaluated to provide HCl salt Form B. Two additional methods were found to provide HCL salt Form B: slurry at room temperature and solid vapor diffusion.

For the slurry experiments at room temperature, approximately 30 mg of amorphous HCl salt of Compound 1 was suspended and stirred in 1.5 mL of IPA/H₂O solutions in 4 mL vials at room temperature. Four IPA/H₂O solutions were used at 20%, 40%, 60%, and 80% IPA respectively. The solids in the slurries were characterized by XRPD after 6 days and identified as HCl Form B.

For the solid vapor diffusion experiments, approximately 30 mg of amorphous HCl salt of Compound 1 was kept in a 4 mL vial which were placed in 20 mL glass vial containing acetonitrile. The solids were characterized by XRPD after 14 days and identified as HCl Form B.

Table 5 describes the XRD pattern of HCl Form B crystalline form of Compound 1 shown in FIG. 2B.

TABLE 5

| Pos. (°2θ) | Intensity | Height (cts) |
|---|---|---|
| 5.6255 | Low | 113.17 |
| 10.0808 | Medium | 705.68 |
| 11.1587 | Low | 120.36 |
| 12.1853 | Medium | 1181.36 |
| 12.3694 | Low | 347.87 |
| 14.3585 | Medium | 1582.51 |
| 14.773 | Medium | 603.33 |
| 15.5104 | Low | 517.4 |
| 16.0316 | Low | 151.38 |
| 16.6685 | Low | 120.81 |
| 17.7134 | Low | 156.21 |
| 18.0423 | Low | 191.89 |
| 18.5916 | Medium | 540.57 |
| 18.91 | Medium | 983.47 |
| 19.7605 | Low | 158.11 |
| 20.4054 | Medium | 591.73 |
| 20.7347 | Low | 114.82 |
| 21.7512 | Low | 95.26 |
| 22.3745 | Medium | 716.17 |
| 22.8168 | Medium | 870.03 |
| 23.1621 | High | 2590.07 |
| 23.8073 | Medium | 862.95 |
| 24.3374 | Low | 301.59 |
| 24.7908 | Low | 1464.1 |
| 25.0264 | Medium | 760.04 |
| 25.4458 | Low | 233.61 |
| 25.8527 | Medium | 579.85 |
| 26.0554 | Medium | 808.07 |
| 26.6502 | Medium | 593.88 |
| 27.3667 | Medium | 531.9 |
| 27.6716 | Medium | 520.49 |
| 28.124 | Low | 489.41 |
| 28.956 | Low | 168.19 |
| 29.6029 | Medium | 620.06 |
| 29.8979 | Medium | 568.06 |
| 30.3345 | Low | 479.71 |
| 31.036 | Low | 318.43 |
| 31.413 | Low | 129.46 |
| 32.1872 | Low | 434.59 |
| 33.044 | Low | 303.07 |
| 33.3108 | Low | 373.75 |
| 33.9668 | Low | 80.35 |
| 34.7938 | Low | 153.22 |
| 35.5083 | Low | 183.4 |

The hygroscopicity of HCl Form B was also evaluated by conducting dynamic vapor sorption experiments. FIG. 2D shows the DVS isotherm of HCl Form B which shows a water uptake of approximately 1.3% at 25° C. between 10% relative humidity and 90% relative humidity. No form change was observed for the sample after DVS evaluation.

### Example 4: Formation of HCl Form E of Compound 1

Various process of preparing crystalline forms of Compound 1 were evaluated to provide HCl salt Form E. Eight methods were found to provide HCl salt Form E: slurry at room temperature, slurry at 50° C., anti-solvent addition, solid vapor diffusion, liquid vapor diffusion, slow evaporation at room temperature, slow cooling, and polymer induced crystallization.

For the slurry experiments at room temperature, approximately 30 mg of amorphous HCl salt of Compound 1 was suspended and stirred in 1.5-2.0 mL of different solvents in 4 mL vials at room temperature. The solids in the slurry were characterized by XRPD after 14 days. Table 6 discloses the parameters resulting in HCl salt Form E for slurry experiments at room temperature.

TABLE 6

| Exp. No. | Solvent (v:v) | Observation | Crystal Form Obtained |
|---|---|---|---|
| 1 | MeOH | Off white solid | HCl salt Form E |
| 2 | NPA | Off white solid | HCl salt Form E |
| 3 | DCM | Off white solid | HCl salt Form E |

For the slurry experiments at 50 CC, approximately 30 mg of amorphous HCl salt of Compound 1 was suspended and stirred in different solvents in 4 mL vials at 50° C. The solids in the slurry were characterized by XRPD after 6 days. Table 7 discloses the parameters resulting in HCl salt Form E for slurry experiments at 50° C.

TABLE 7

| Exp. | | | Crystal Form |
|---|---|---|---|
| No. | Solvent (v:v) | Observation | Obtained |
| 4 | EtOH | Off white solid | HCl salt Form E |
| 5 | ACN | Off white solid | HCl salt Form E |
| 6 | IPA/Tolune (1:1) | Off white solid | HCl salt Form E |
| 7 | MeOH | Off white solid | HCl salt Form E |
| 8 | MeOH/H₂O (1:1) | Off white solid | HCl salt Form E |
| | THF/n-hepatane (1:1) | Off white solid | HCl salt Form E |

For the anti-solvent addition experiments, approximately 30 mg of amorphous HCl salt of Compound 1 was dissolved in different solvents to obtain saturated solutions and anti-solvents were added in amounts of up to 20 times in volume. The obtained solids were characterized by XRPD. Table 8 discloses the parameters resulting in HCl Form E of anti-solvent addition experiments.

TABLE 8

| Exp. | Solvent | Anti- | | Crystal Form |
|---|---|---|---|---|
| No. | (v:v) | solvent | Observation | Obtained |
| 9 | MeOH | MTBE | Off white solid | HCl salt Form E |
| 10 | MeOH | ACN | Off white solid | HCl salt Form E |
| 11 | MeOH | Heptane | Off white solid | HCl salt Form E |

For the solid vapor diffusion experiments, approximately 30 mg of amorphous HCl salt of Compound 1 was kept in 4 mL vials which were placed in 20 mL glass vials containing different solvents. The solids were characterized by XRPD after 14 days. Table 9 discloses the parameters resulting in HCl salt Form E of solid vapor diffusion experiments.

TABLE 9

| Exp. No. | Solvent (v:v) | Observation | Crystal Form Obtained |
|---|---|---|---|
| 12 | EtOH | Off white solid | HCl salt Form E |

For the liquid vapor diffusion experiments, approximately 30 mg of amorphous HCl salt of Compound 1 was dissolved in different solvents to obtain saturated solutions in 4 mL vials which were placed in 20 mL vials containing anti-solvents. The obtained solids were characterized by XRPD after 12 days. Table 10 discloses the parameters resulting in HCl salt Form E of liquid vapor diffusion experiments.

TABLE 10

| Exp. | Solvent | Anti- | | Crystal Form |
|---|---|---|---|---|
| No. | (v:v) | solvent | Observation | Obtained |
| 13 | MeOH | CPME | Off white solid | HCl salt Form E |
| 14 | MeOH | ACN | Off white solid | HCl salt Form E |
| 15 | MeOH | IPAC | Off white solid | HCl salt Form E |

For the slow evaporation experiments, evaporation occurred at either room temperature of 50° C. To obtain saturated solutions, approximately 30 mg of amorphous HCl salt of Compound 1 was dissolved in different solvents in 4 mL vials. The vials were covered with paraffin film with 3-5 holes and placed at room temperature or 50° C. for evaporation. The obtained solids were characterized by XRPD. Table 11 discloses the parameters resulting in HCl salt Form E of slow evaporation experiments.

TABLE 11

| Exp. | Solvent | Temp. | | Crystal Form |
|---|---|---|---|---|
| No. | (v:v) | (° C.) | Observation | Obtained |
| 16 | MeOH | RT | Off white solid | HCl salt Form E |
| 17 | IPA | 50 | Off white solid | HCl salt Form E |
| 18 | 1-BuOH | 50 | Off white solid | HCl salt Form E |

For the slow cooling experiments, approximately 30 mg of amorphous HCl salt of Compound 1 was dissolved in different solvents to obtain saturated solutions in 4 mL vials at 55° C. using a hot plate. The solutions were slowly cooled down to room temperature using the following ramp: 55° C. for 3 hours, 40° C. for 4 hours, 25° C. for 4 hours, 15° C. for 5 hours, and 10° C. for 5 hours. The obtained solids were characterized by XRPD. Table 12 discloses the parameters resulting in HCl salt Form E of slow cooling experiments.

TABLE 12

| Exp. No. | Solvent (v:v) | Observation | Crystal Form Obtained |
|---|---|---|---|
| 19 | MeOH | Off white solid | HCl salt Form E |
| 20 | EtOH | Off white solid | HCl salt Form E |

For the polymer induced crystallization experiments, approximately 30 mg of amorphous HCl salt of Compound 1 was dissolved in different solvents to create saturated solutions. Either polyvinylpyrrolidone (PVP), polyethylene glycol (PEO), polyvinyl alcohol (PVA), or hydroxypropyl methylcellulose (HPMC) were added to the saturated solutions to induce heteronucleation. The obtained solids were characterized by XRPD after 12 days. Table 13 discloses the parameters resulting in HCl salt Form E of polymer induced crystallization experiments.

TABLE 13

| Exp. | Solvent | | | Crystal Form |
|---|---|---|---|---|
| No. | (v:v) | Polymer | Observation | Obtained |
| 21 | MeOH | PVP | Off white solid | HCl salt Form E |

HCl salt Form E was characterized by various methods including XRPD, DSC, TGA, DVS, and ¹H-NMR. The results of the characterization of summarized in Table 14 and shown in FIGS. 3A-3D.

TABLE 14

| Sample | HCl Form E |
|---|---|
| Crystallinity by XRPD | High |
| Wt. Loss in TGA (%, ° C.) | 1.06 (150) |
| Endo. in DSC (peak, ° C.) | 239.31 |
| Heat of fusion (J/g) | 98.84 |
| Hygroscopiscity (25° C./80% RH) | 2.56% (slightly |
| | hygroscopic) |
| Form | Likely Anhydrate |

Table 15 describes the XRD pattern of HCl Form E crystalline form of Compound 1 shown in FIG. 4A.

TABLE 15

| Pos. (°2θ) | Intensity | Height (cts) |
|---|---|---|
| 5.5035 | Low | 216.82 |
| 9.7277 | High | 1186.09 |
| 10.9584 | Low | 269.4 |
| 12.0773 | Medium | 815.2 |
| 14.2728 | High | 1057.29 |
| 14.9884 | Low | 399.31 |
| 15.8331 | Low | 113.07 |
| 17.0744 | Low | 157.37 |
| 17.9635 | Low | 174.83 |
| 18.4651 | Medium | 463.44 |
| 19.6489 | Low | 114.37 |
| 20.025 | Low | 263.63 |
| 22.3453 | Medium | 822.13 |
| 22.8235 | High | 1838.83 |
| 23.1975 | Low | 386.45 |
| 23.7047 | Low | 205.91 |
| 24.5443 | Medium | 881.07 |
| 25.435 | Low | 363.87 |
| 26.1019 | Low | 256.23 |
| 26.9867 | Low | 267.5 |
| 27.7112 | Low | 242.21 |
| 29.2508 | Low | 201.42 |
| 29.6655 | Low | 295.37 |
| 30.9699 | Low | 209.36 |
| 32.9353 | Low | 114.51 |

The hygroscopicity of HCl Form E was also evaluated by conducting dynamic vapor sorption experiments. FIG. 3C shows the DVS isotherm of HCl Form E which shows a water uptake of approximately 2.56% at 25° C. between 0% relative humidity and 80% relative humidity. No form change was observed for the sample after DVS evaluation.

### Example 5: Formation of HCl Form F of Compound 1

HCl Form F of Compound 1 was formed by liquid vapor diffusion. The amorphous HCl salt of Compound 1 (30 mg) was dissolvent in NMP to obtain saturated solutions of 4 mL vials which were places in 20 mL glass vials containing anti-solvents (heptane and MTBE). The obtained solids were there then characterized after 12 days by XRPD, DSC, TGA, and ¹H-NMR. The crystals produced were an off-white solid. The results of the characterization of summarized in Table 16 and shown in FIGS. 4A-4C.

TABLE 16

| Sample | HCl Form F |
|---|---|
| Crystallinity by XRPD | High |
| Wt. Loss in TGA (%, ° C.) | 9.52 (70) |
| Endo. in DSC (peak, ° C.) | 48.30 |
| | 97.28 |
| | 162.21 |
| Heat of fusion (J/g) | 98.15.03 |
| | 21.25 |
| | 116.92 |
| Form | NMP solvate |

Table 17 describes the XRD pattern of HCl Form F crystalline form of Compound 1 shown in FIG. 5A.

TABLE 17

| Pos. (°2θ) | Intensity | Height (cts) |
|---|---|---|
| 4.2827 | Medium | 794.01 |
| 6.2827 | High | 980.36 |
| 7.8816 | Medium | 788.4 |
| 9.681 | Low | 145.17 |
| 11.1865 | Low | 70.63 |
| 12.5403 | Low | 261.97 |
| 12.7783 | Low | 159.62 |
| 13.5785 | Low | 196.59 |
| 15.2143 | Low | 151.27 |
| 15.6094 | Low | 172.86 |
| 17.0461 | Low | 174.81 |
| 17.5382 | Low | 286.68 |
| 18.8367 | High | 1286.99 |
| 19.3909 | Low | 204.02 |
| 20.3531 | High | 1311.31 |
| 21.326 | Low | 147.67 |
| 21.8359 | Low | 255.56 |
| 23.2695 | Low | 360.06 |
| 23.6941 | High | 1187.91 |
| 23.767 | High | 1273.91 |
| 24.5981 | Low | 57.59 |
| 25.1852 | Medium | 758.98 |
| 25.4916 | High | 1024.74 |
| 26.0928 | Low | 173.23 |
| 26.7084 | High | 1568.98 |
| 27.343 | Medium | 397.42 |
| 28.2501 | Medium | 620.47 |
| 28.3237 | Medium | 431.28 |
| 28.8055 | Low | 138.27 |
| 29.3794 | Low | 142.74 |
| 30.0352 | Low | 205.95 |
| 30.1599 | Medium | 372.39 |
| 30.411 | Low | 138.96 |
| 31.4742 | Low | 159.92 |
| 31.5999 | Low | 120.64 |
| 32.5745 | Low | 89.84 |
| 33.5893 | Low | 78.59 |
| 34.4552 | Low | 135.31 |
| 34.7729 | Medium | 426.99 |
| 37.2647 | Low | 119.29 |
| 39.2069 | Low | 128.97 |

### Example 6: Formation of HCl Form G of Compound 1

HCl Form G of Compound 1 was formed by solid vapor diffusion. The amorphous HCl salt of Compound 1 (30 mg) was kept in a 4 mL vial which were places in 20 mL glass vials containing NMP. The obtained solids were there then characterized after 14 days by XRPD, DSC, TGA, and ¹H-NMR. The crystals produced were an off-white solid. The results of the characterization of summarized in Table 18 and shown in FIGS. 5A-5C.

TABLE 18

| Sample | HCl Form G |
|---|---|
| Crystallinity by XRPD | Moderate |
| Wt. Loss in TGA (%, ° C.) | 5.48 (110) |
| Endo. in DSC (peak, ° C.) | 61.86 |
| | 157.17 |
| Heat of fusion (J/g) | 26.78 |
| | 68.55 |
| Form | NMP solvate |

Table 19 describes the XRD pattern of HCl Form G crystalline form of Compound 1 shown in FIG. 6A.

TABLE 19

| Pos. (°2θ) | Intensity | Height (cts) |
|---|---|---|
| 4.2313 | Low | 45.08 |
| 5.0139 | Low | 33.32 |
| 6.2791 | High | 509.44 |
| 7.8695 | Low | 185.89 |
| 11.1952 | Low | 70.2 |
| 11.7674 | Low | 57.12 |
| 12.537 | Medium | 246.48 |
| 13.7817 | Low | 117.78 |
| 14.382 | Medium | 206.84 |
| 14.5205 | Medium | 205.83 |
| 15.1984 | Medium | 333.77 |
| 15.8969 | Low | 75.21 |
| 16.5851 | Medium | 252.37 |
| 17.401 | Medium | 344.06 |
| 17.7496 | Low | 169.47 |
| 18.4571 | Low | 156.84 |
| 18.8409 | High | 587.41 |
| 19.0963 | Low | 143.73 |
| 19.5272 | Low | 138.82 |
| 20.3541 | High | 430.9 |
| 21.5134 | Medium | 294.96 |
| 21.8209 | Low | 166.85 |
| 22.3447 | High | 419.28 |
| 22.5708 | Medium | 257.29 |
| 23.1338 | Low | 76.52 |
| 23.7725 | High | 675.54 |
| 24.45 | Low | 138.09 |
| 25.1898 | Medium | 376.35 |
| 25.5119 | Medium | 237.56 |
| 25.9501 | Medium | 356.94 |
| 26.3533 | Medium | 319.07 |
| 26.7124 | High | 583.91 |
| 26.9575 | Low | 191.36 |
| 27.3731 | Low | 77.77 |
| 27.9972 | Low | 73.47 |
| 28.3204 | Low | 149.54 |
| 28.66 | Low | 167.43 |
| 29.4873 | Low | 181.32 |
| 30.1727 | Low | 162.96 |

### Example 7: Competitive Slurry Experiments

Table 20 summarizes the results from competitive slurry experiments at different conditions to explore the relative stability of HCl Form A, HCl Form B, and HCl Form E. Experiments CS-1 to CS-6 were conducted at room temperature with a mixture of HCl Form E (30 mg), of HCl Form A (5 mg), and of HCl Form B (5 mg) suspended in solutions with different water activities (α_{w}). Experiment CS-7 was set up with only of HCl Form E suspended in pure water at room temperature. Experiment CS-8 was conducted with of HCl Form A and of HCl Form B in pure water at 60° C. Experiment CS-9 was conducted with amorphous HCl salt in a EtOH/water solution (40:60 v/v) at room temperature. Pattern A was observed under certain conditions during competitive slurry experiments. Pattern A was confirmed to be racemate by chiral HPLC analysis.

TABLE 20

| | | | | Crystal |
|---|---|---|---|---|
| Exp.ID | Solvent | Duration | | Form |
| CS-1 | MeOH (α_{w} = | 2 | d | Form E |
| | 0.0) at 25° C. | 6 | d | Form E |
| | | 10 | d | Form E |
| CS-2 | MeOH/H₂O (α_{w} = | 2 | d | Form E |
| | 0.2) at 25° C. | 6 | d | Form E |
| | | 10 | d | Form E |
| CS-3 | MeOH/H₂O (α_{w} = | 3 | d | Pattern A |
| | 0.4) at 25° C. | 6 | d | Form B + |
| | | | | Pattern A |
| | | 10 | d | Form B + |
| | | | | Pattern A |
| | | | | (mainly |
| | | | | Form B) |
| | | 20 | d | Form B + |
| | | | | Pattern A |
| | | | | (mainly |
| | | | | Form B) |
| CS-4 | MeOH/H₂O (α_{w} = | 6 | d | Pattern A |
| | 0.6) at 25° C. | 10 | d | Form B + |
| | | | | Pattern A |
| | | | | (mainly |
| | | | | Form B) |
| | | 20 | d | Form B + |
| | | | | Pattern A |
| | | | | (mainly |
| | | | | Form B) |
| CS-5 | MeOH/H₂O (α_{w} = | 6 | d | Pattern A |
| | 0.8) at 25° C. | 10 | d | Form B + |
| | | | | Pattern A |
| | | | | (mainly |
| | | | | Form B) |
| | | 20 | d | Pattern A |
| CS-6 | H₂O (α_{w} = | 3 | d | Form A + |
| | 1.0) at 25° C. | | | Form B |
| | | 6 | d | Form A + |
| | | | | Form B |
| | | 10 | d | Form A + |
| | | | | Form B |
| | | 20 | d | Form B |
| CS-7 | H₂O (starting | 4 | d | Form B |
| | with Type E only) | | | |
| | at 25° C. | 8 | d | Form B |
| CS-8 | H₂O (Form 1 and | 1 | d | Form B |
| | Form 2 at 60° C.) | 6 | d | Form B + |

| | | | |
|---|---|---|---|
| | | | Pattern A |
| CS-9 | EtOH/H₂O (40:60 | 1d (a | Pattern A |
| | v/v, starting | clear solution | |
| | with amorphous | was obtained | |
| | HCl salt at | before | |
| | at 25° C.) | precipitation) | |

### Example 8: Pre-Clinical Bioperformance

The pharmacokinetics of different crystalline forms of Compound 1 was investigated following oral administration of Free Base Form A, HCl Form A, and HCl Form B of Compound 1 in male beagle dogs at a dose of 100 mg/dog (~Approximately 10 mg per kg). The substances were filled into Empty HPMC Capsules after correcting for potency and administered to dogs pre-treated with either pentagastrin or famotidine along with 40 mL of water. Pentagastrin enhances gastric secretions whereas famotidine neutralizes gastric sections. Therefore, pentagastrin and famotidine pre-treatments were used to simulate the impact of gastric pH on bioavailability and reduce the variability in gastric pH between dogs.

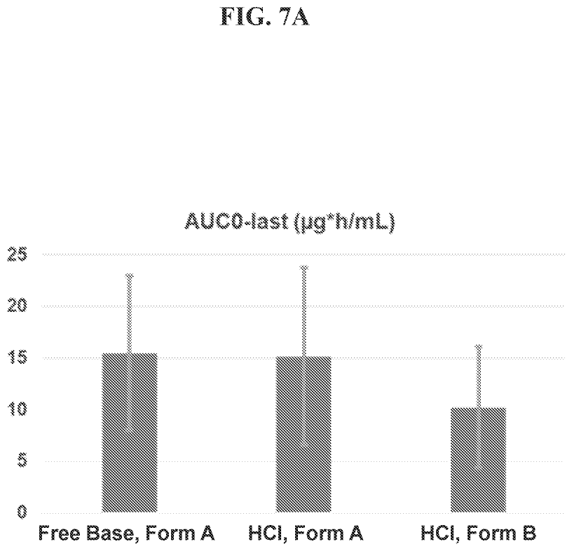

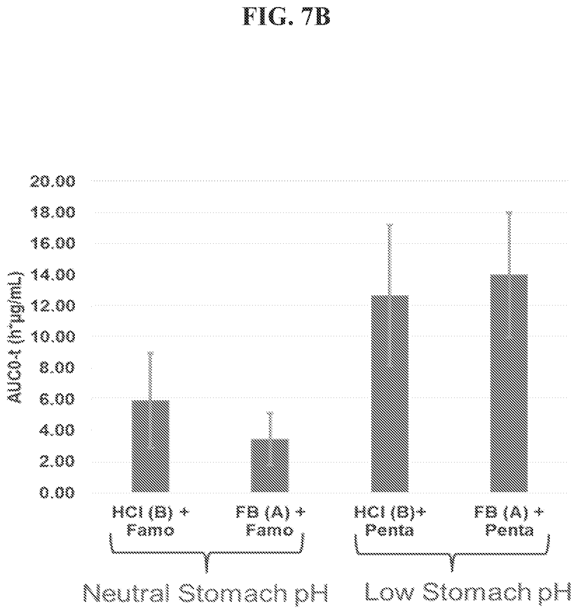

Plasma samples were collected at pre-defined intervals and analyzed for the concentration of Compound 1. The plasma concentration versus time data was analyzed by non-compartmental approaches using Win Nonlin software program to estimate PK parameters such as Cₘₐₓ, Tₘₐₓ, AUC₀₋ₜ and AUC_{0-inf}. These results are shown in FIGS. 7A and 7B.

From the results, it was found that HCl Form A was less stable than HCl Form B and that HCl salt exhibited a common ion effect in the gastric media which may have caused high variability in pentagastrin-treated dogs. Additionally, Free Bass Form A has an equivalent exposure to HCl salt when the particle size was normalized. And additionally, for both HCl forms tested there was marked differences in the bioavailability between dogs treated with pentagastrin and famotidine, which suggests a possible food effect for both forms.

### Example 9: Excipient Compatibility

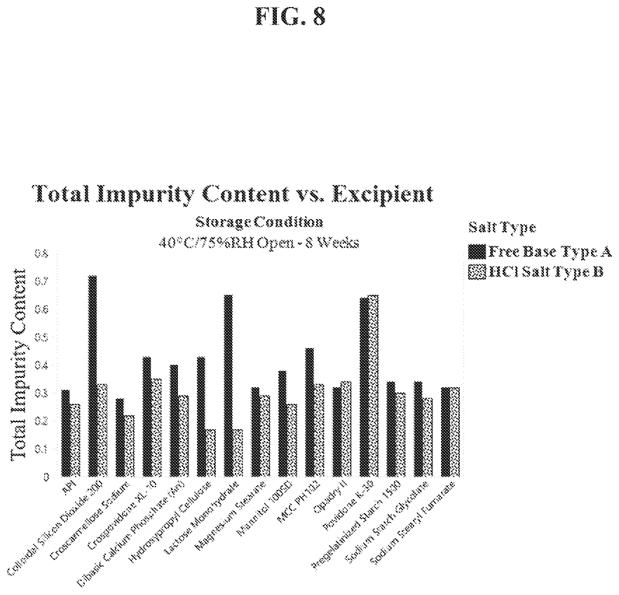

The compatibility of both the Free Base Form A and HCl Form B of Compound 1 with multiple excipients commonly used in Oral Solid Dosage (OSD) formulation development was evaluated. Binary mixtures of samples were created by mixing the substance and the excipients (1:10 Compound to Excipients for fillers and 1:1 for all other excipients) using a mortar and pestle and the resultant mixture was accurately weighed and transferred into sample vials. The excipients used were as follows: colloidal silicon dioxide 200, croscarmellose sodium, crospovidone XL-10, dibasic calcium phosphate (an), hydroxypropyl cellulose, lactose monohydrate, magnesium stearate, mannitol 100SD, MCC PH102, Opadry™ II, povidone K-30, pregelatinized starch 1500, sodium starch glycolate, and sodium stearyl fumarate. The sample vials were set down at 40° C./75% RH condition for 8 weeks in open condition before analyzing their impurity levels using HPLC. The results of the total impurity content for these samples are shown in FIG. 8 and are compared to Free Base Form A and HCl Form B of Compound 1 without any excipients.

From FIG. 8, it can be seen that both Free Base Form A and HCl Form B of Compound 1 exhibit good compatibility with excipients. Additionally, Free Base Form A was more sensitive to colloidal silicon dioxide and lactose monohydrate than HCl Form B. Although the risk of chemical stability is slightly higher for the Free Base Form A and HCl Form B, it is still manageable by influencing the choice of excipients selected.

### Example 10: Manufacturability and Physical Stability

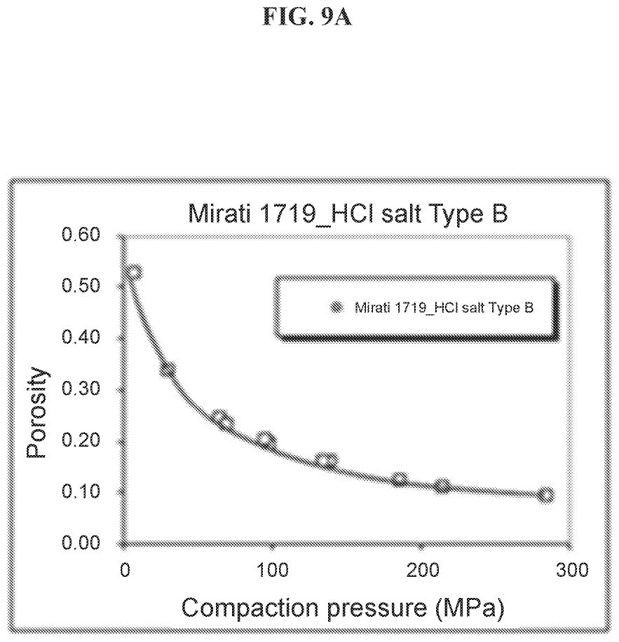

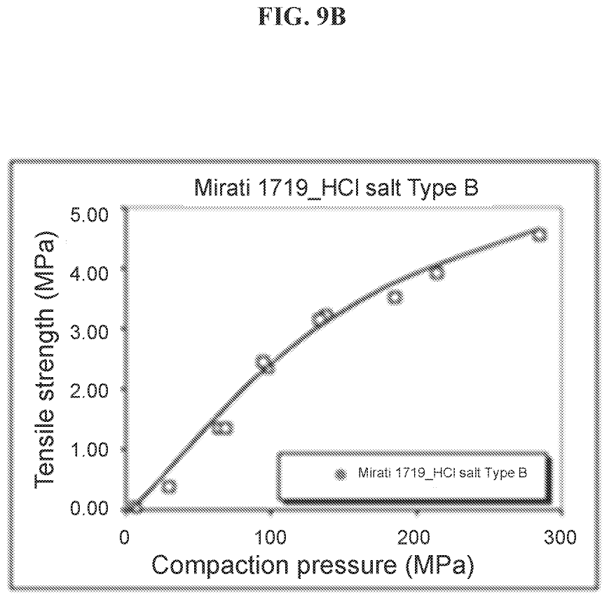

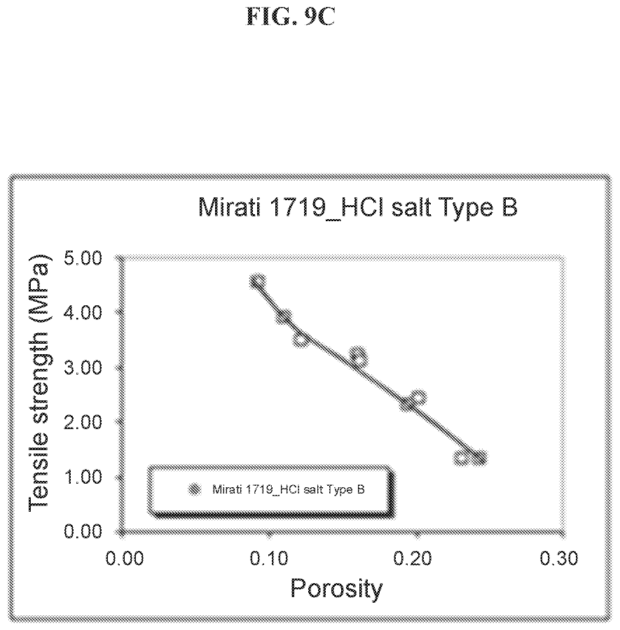

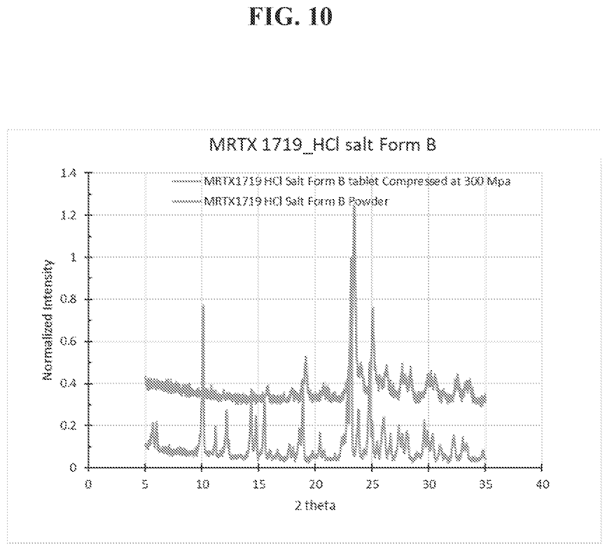

The compressibility (i.e., porosity vs compaction pressure), tabletability (i.e., tensile strength vs compaction pressure), and compatibility (i.e. tensile strength vs porosity) of HCl Form B of Compound 1 was measured. The results of these tests are shown in FIGS. 9A-9C. Additionally, the phase stability of the HCl Form B of Compound 1 at a compaction pressure of 300 MPa was investigated by XRD. The XRD patterns were compared with the uncompressed compound. X-ray powder diffractograms were obtained on a wide-angle X-ray diffraction instrument (X'Pert Pro; PANalytical Inc., West Borough, MA) using Cu Ká radiation. The voltage and current applied were 45 kV and 40 mA, respectively. Each measurement was performed with a step size of 0.0167° in the 2-theta range of 5-35° and a dwell time of 0.4 s. These results are shown in FIG. 10.

The HCl Form B exhibited good compressibility, tabletability, and compatibility at high pressures, although a loss of crystallinity was observed at high compaction pressure.

### Example 11: Intrinsic Dissolution Rate (IDR) and Drug in Capsule Dissolution in Fasted Gastric Simulated Fluid (FaSSGF) and Fasted Simulated Intestinal Fluid (FaSSIF)

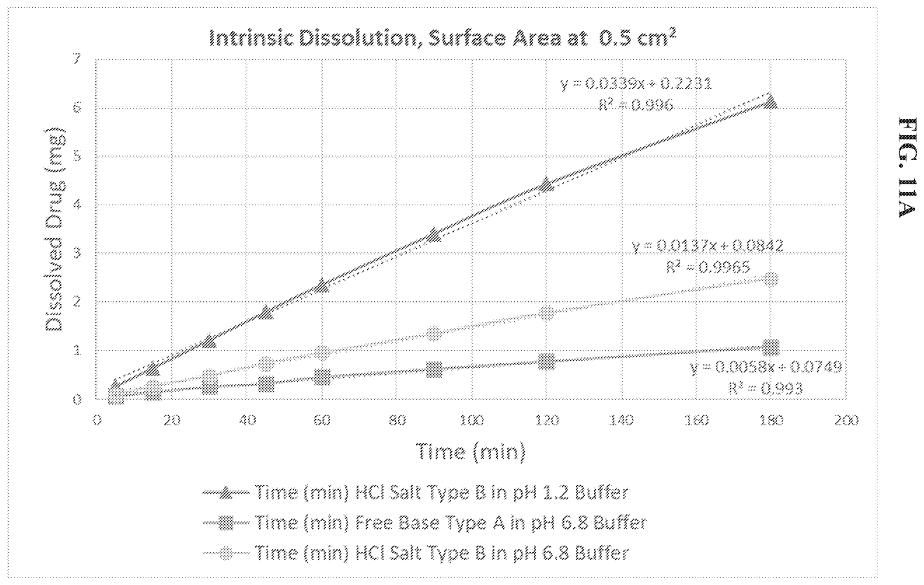

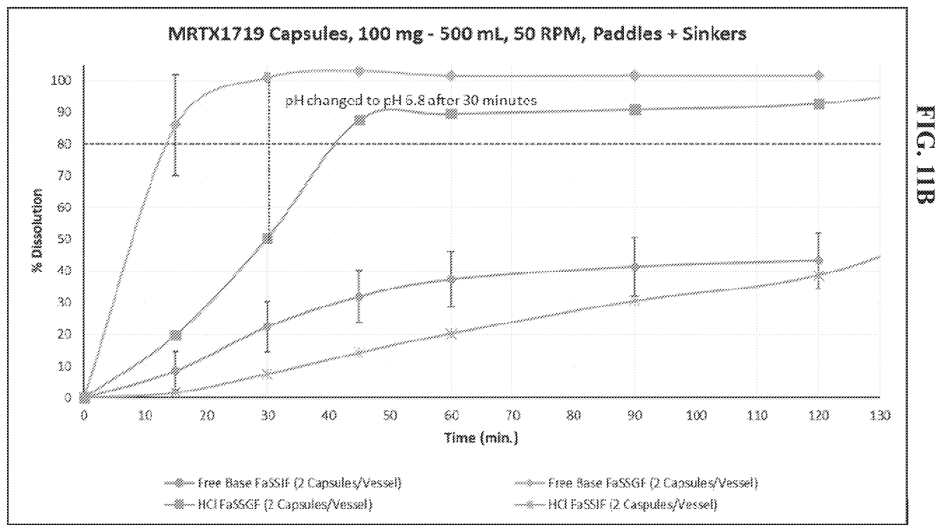

The intrinsic dissolution rate (IDR) was determined by measuring the amount of dissolved Free Base Form A and HCl Form B of Compound 1 in the buffer and then linear curve of dissolved compounds versus time was constructed. The IDR rate was calculated using the slope of the linear curve divided by the surface area of tablet (0.5 cm²). Free Base Form A and HCl Form B of Compound 1 were pressed into a tablet with 0.8 cm diameter at 14 MPa, respectively. Capsules of HCl Form B were stirred in pH 1.2 and pH 6.8 buffer at 37° C. with 50 rpm in a USP Type II dissolution apparatus with sinkers, while capsules of Free Base Form A were stirred in pH 6.8 buffer under the same conditions. 0.6 mL liquid was removed with a syringe at each time point and filtered. The concentration of filtrates was analyzed by HPLC. The IDR results are shown in FIG. 11A and the dissolution in capsules is shown in FIG. 11B.

The Free Base Form A exhibited faster dissolution in gastric media due to common ion effect slowing dissolution of HCl Form B. In intestinal media, HCl Form B has slightly faster dissolution rate than Free Base Form A, however it is observed that Free Base Form A is able to maintain super saturation in intestinal fluid when the pH is shifted from gastric to intestinal after 30 minutes.

### ITEMIZED LIST OF EMBODIMENTS

Embodiment 1. A crystalline form of 2-(4-(4-(aminomethyl)-1-oxo-1,2-dihydrophthalazin-6-yl)-1-methyl-1h-pyrazol-5-yl)-4-chloro-6-cyclopropoxy-3-fluorobenzonitrile hydrochloride salt having an endothermic differential scanning calorimetric (DSC) peak temperature within ±2% of 118° C.

Embodiment 2. The crystalline form of embodiment 1, wherein the endothermic DSC peak temperature is within ±1% of 118° C.

Embodiment 3. The crystalline form of embodiment 1, wherein the endothermic DSC peak temperatures is within ±0.5% of 118° C.

Embodiment 4. The crystalline form of embodiment 1, wherein the crystalline form has an endothermic DSC peak temperature within ±2% of 211° C.

Embodiment 5. The crystalline form of embodiment 4, wherein the endothermic DSC peak temperature is within ±1% of 211° C.

Embodiment 6. The crystalline form of embodiment 4, wherein the endothermic DSC peak temperature is within ±0.5% of 211° C.

Embodiment 7. A crystalline form of 2-(4-(4-(aminomethyl)-1-oxo-1,2-dihydrophthalazin-6-yl)-1-methyl-1h-pyrazol-5-yl)-4-chloro-6-cyclopropoxy-3-fluorobenzonitrile hydrochloride salt having an X-ray powder diffraction (XRPD) pattern comprising a peak at a two-theta angle of 6.8°±0.2°.

Embodiment 8. The crystalline form of embodiment 7, wherein the crystalline form has an X-ray powder diffraction (XRPD) pattern comprising a peak at a two-theta angle of 7.4°±0.2°.

Embodiment 9. The crystalline form of embodiment 7, wherein the crystalline form has an X-ray powder diffraction (XRPD) pattern comprising a peak at a two-theta angle of 13.9°±0.2°.

Embodiment 10. The crystalline form of embodiment 7, wherein the crystalline form has an X-ray powder diffraction (XRPD) pattern comprising a peak at a two-theta angle of 23.9°±0.2°.

Embodiment 11. The crystalline form of embodiment 7, wherein the crystalline form has an X-ray powder diffraction (XRPD) pattern comprising a peak at a two-theta angle of 26.2°±0.2°.

Embodiment 12. The crystalline form of embodiment 7, wherein the crystalline form has an X-ray powder diffraction (XRPD) pattern comprising peaks at a two-theta angle of 6.8°±0.2°, 7.4°±0.2°, 13.9°±0.2°, 23.9°±0.2°, and 26.2°±0.2°.

Embodiment 13. A crystalline form of 2-(4-(4-(aminomethyl)-1-oxo-1,2-dihydrophthalazin-6-yl)-1-methyl-1h-pyrazol-5-yl)-4-chloro-6-cyclopropoxy-3-fluorobenzonitrile hydrochloride salt having an X-ray powder diffraction pattern substantially shown in FIG. 1B.

Embodiment 14. The crystalline form of any of embodiments 1-13, wherein the crystalline form is a tri-hydrate.

Embodiment 15. The crystalline forma of any of embodiments 1-14, wherein the crystalline form has a thermal gravimetric analysis (TGA) plot comprising a mass loss of about 9.7% when heated from about 25° C. to about 150° C.

Embodiment 16. The crystalline form of any of embodiments 1-15, wherein the crystalline form has a purity of at least 97% by weight of 2-(4-(4-(aminomethyl)-1-oxo-1,2-dihydrophthalazin-6-yl)-1-methyl-1h-pyrazol-5-yl)-4-chloro-6-cyclopropoxy-3-fluorobenzonitrile hydrochloride salt.

Embodiment 17. The crystalline form of any of embodiments 1-16, wherein the crystalline form has a purity of at least 98% by weight of 2-(4-(4-(aminomethyl)-1-oxo-1,2-dihydrophthalazin-6-yl)-1-methyl-1h-pyrazol-5-yl)-4-chloro-6-cyclopropoxy-3-fluorobenzonitrile hydrochloride salt.

Embodiment 18. A pharmaceutical composition comprising the crystalline form of any of embodiments 1-17 and a pharmaceutically acceptable carrier.

Embodiment 19. A crystalline form of 2-(4-(4-(aminomethyl)-1-oxo-1,2-dihydrophthalazin-6-yl)-1-methyl-1h-pyrazol-5-yl)-4-chloro-6-cyclopropoxy-3-fluorobenzonitrile hydrochloride salt having an endothermic differential scanning calorimetric (DSC) peak temperature within ±2% of 97° C.

Embodiment 20. The crystalline form of embodiment 19, wherein the endothermic DCS peak temperature is within ±1% of 97° C.

Embodiment 21, The crystalline form of embodiment 19, wherein the endothermic DCS peak temperature is within ±0.5% of 97° C.

Embodiment 22. The crystalline form of embodiment 19, wherein the endothermic crystalline form has a DSC peak temperature within ±2% of 245° C.

Embodiment 23. The crystalline form of embodiment 19, wherein the endothermic DCS peak temperature is within ±1% of 245° C.

Embodiment 24. The crystalline form of embodiment 19, wherein the endothermic DCS peak temperature is within ±0.5% of 245° C.

Embodiment 25. A crystalline form of 2-(4-(4-(aminomethyl)-1-oxo-1,2-dihydrophthalazin-6-yl)-1-methyl-1h-pyrazol-5-yl)-4-chloro-6-cyclopropoxy-3-fluorobenzonitrile hydrochloride salt having an X-ray powder diffraction (XRPD) pattern comprising a peak at a two-theta angle of 12.2°±0.2°.

Embodiment 26. The crystalline form of embodiment 25, wherein the crystalline form has an X-ray powder diffraction (XRPD) pattern comprising a peak at a two-theta angle of 14.3±0.2°.

Embodiment 27. The crystalline form of embodiment 25, wherein the crystalline form has an X-ray powder diffraction (XRPD) pattern comprising a peak at a two-theta angle of 18.9°±0.2°.

Embodiment 28. The crystalline form of embodiment 25, wherein the crystalline form has an X-ray powder diffraction (XRPD) pattern comprising a peak at a two-theta angle of 23.2°±0.2°.

Embodiment 29. The crystalline form of embodiment 25, wherein the crystalline form has an X-ray powder diffraction (XRPD) pattern comprising a peak at a two-theta angle of 26.0°±0.2°.

Embodiment 30. The crystalline form of embodiment 25, wherein the crystalline form has an X-ray powder diffraction (XRPD) pattern comprising peaks at a two-theta angle of 12.2°±0.2°, 14.3°±0.2°, 18.9°±0.2°, 23.2°±0.2°, and 26.0°±0.2°.

Embodiment 31. A crystalline form of 2-(4-(4-(aminomethyl)-1-oxo-1,2-dihydrophthalazin-6-yl)-1-methyl-1h-pyrazol-5-yl)-4-chloro-6-cyclopropoxy-3-fluorobenzonitrile hydrochloride salt having an X-ray powder diffraction pattern substantially shown in FIG. 2B.

Embodiment 32. The crystalline form of any of embodiments 19-31, wherein the crystalline form is a mono-hydrate.

Embodiment 33. The crystalline form of any of embodiments 19-32, wherein the crystalline form has a thermal gravimetric analysis (TGA) plot comprising a mass loss of 3.6% when heated from about 25° C. to about 150° C.

Embodiment 34. The crystalline form of any of embodiments 19-33, wherein the crystalline form has a purity of at least 97% by weight of 2-(4-(4-(aminomethyl)-1-oxo-1,2-dihydrophthalazin-6-yl)-1-methyl-1h-pyrazol-5-yl)-4-chloro-6-cyclopropoxy-3-fluorobenzonitrile hydrochloride salt.

Embodiment 35. The crystalline form of any of embodiments 19-34, wherein the crystalline form has a purity of at least 98% by weight of 2-(4-(4-(aminomethyl)-1-oxo-1,2-dihydrophthalazin-6-yl)-1-methyl-1h-pyrazol-5-yl)-4-chloro-6-cyclopropoxy-3-fluorobenzonitrile hydrochloride salt.

Embodiment 36. A pharmaceutical composition comprising the crystalline form of any of embodiments 19-35 and a pharmaceutically acceptable carrier.

Embodiment 37. A crystalline form of 2-(4-(4-(aminomethyl)-1-oxo-1,2-dihydrophthalazin-6-yl)-1-methyl-1h-pyrazol-5-yl)-4-chloro-6-cyclopropoxy-3-fluorobenzonitrile hydrochloride salt having an endothermic differential scanning calorimetric (DSC) peak temperature within ±2% of 239° C.

Embodiment 38. The crystalline form of embodiment 37, wherein the endothermic DSC peak temperature is within ±1% of 239° C.

Embodiment 39. The crystalline form of embodiment 37, wherein the endothermic DSC peak temperature is within ±0.5% of 239° C.

Embodiment 40. A crystalline form of 2-(4-(4-(aminomethyl)-1-oxo-1,2-dihydrophthalazin-6-yl)-1-methyl-1h-pyrazol-5-yl)-4-chloro-6-cyclopropoxy-3-fluorobenzonitrile hydrochloride salt having an X-ray powder diffraction (XRPD) pattern comprising a peak at a two-theta angle of 9.7°±0.2°.

Embodiment 41. The crystalline form of embodiment 40, wherein the crystalline form has an X-ray powder diffraction (XRPD) pattern comprising a peak at a two-theta angle of 12.10°±0.2°.

Embodiment 42. The crystalline form of embodiment 40, wherein the crystalline form has an X-ray powder diffraction (XRPD) pattern comprising a peak at a two-theta angle of 14.3°±0.2°.

Embodiment 43. The crystalline form of embodiment 40, wherein the crystalline form has an X-ray powder diffraction (XRPD) pattern comprising a peak at a two-theta angle of 22.3°±0.2°.

Embodiment 44. The crystalline form of embodiment 40, wherein the crystalline form has an X-ray powder diffraction (XRPD) pattern comprising a peak at a two-theta angle of 22.8°±0.2°.

Embodiment 45. The crystalline form of embodiment 40, wherein the crystalline form has an X-ray powder diffraction (XRPD) pattern comprising a peak at a two-theta angle of 24.5°±0.2°.

Embodiment 46. The crystalline form of embodiment 40, wherein the crystalline form has an X-ray powder diffraction (XRPD) pattern comprising peaks at a two-theta angle of 9.7°±0.2°, 12.1°±0.2°, 14.3°±0.2°, 22.3°±0.2°, 22.8°±0.2°, and 24.5°±0.2°.

Embodiment 47. A crystalline form of 2-(4-(4-(aminomethyl)-1-oxo-1,2-dihydrophthalazin-6-yl)-1-methyl-1h-pyrazol-5-yl)-4-chloro-6-cyclopropoxy-3-fluorobenzonitrile hydrochloride salt having an X-ray powder diffraction pattern substantially shown in FIG. 3B.

Embodiment 48. The crystalline form of any of embodiments 37-47, wherein the crystalline form is an anhydrate.

Embodiment 49. The crystalline form of any of embodiments 37-48, wherein the crystalline form has a thermal gravimetric analysis (TGA) plot comprising a mass loss of about 1.0% when heated from about 25° C. to about 150° C.

Embodiment 50. The crystalline form of any of embodiments 37-49, wherein the crystalline form has a purity of at least 97% by weight of 2-(4-(4-(aminomethyl)-1-oxo-1,2-dihydrophthalazin-6-yl)-1-methyl-1h-pyrazol-5-yl)-4-chloro-6-cyclopropoxy-3-fluorobenzonitrile hydrochloride salt.

Embodiment 51. The crystalline form of any of embodiments 37-50, wherein the crystalline form has a purity of at least 98% by weight of 2-(4-(4-(aminomethyl)-1-oxo-1,2-dihydrophthalazin-6-yl)-1-methyl-1h-pyrazol-5-yl)-4-chloro-6-cyclopropoxy-3-fluorobenzonitrile hydrochloride salt.

Embodiment 52. A pharmaceutical composition comprising the crystalline form of any of embodiments 37-51 and a pharmaceutically acceptable carrier.

Embodiment 53. A crystalline form of 2-(4-(4-(aminomethyl)-1-oxo-1,2-dihydrophthalazin-6-yl)-1-methyl-1h-pyrazol-5-yl)-4-chloro-6-cyclopropoxy-3-fluorobenzonitrile hydrochloride salt having an endothermic differential scanning calorimetric (DSC) peak temperature within ±2% of 15° C.

Embodiment 54. The crystalline form of embodiment 53, wherein the endothermic DSC peak temperature is within ±1% of 15° C.

Embodiment 55. The crystalline form of embodiment 53, wherein the endothermic DSC peak temperature is within ±0.5% of 15° C.

Embodiment 56. The crystalline form of embodiment 53, wherein the crystalline form has an endothermic DSC peak temperature within ±2% of 97° C.

Embodiment 57. The crystalline form of embodiment 56, wherein the endothermic DSC peak temperature is within ±1% of 97° C.

Embodiment 58. The crystalline form of embodiment 56, wherein the endothermic DSC peak temperature is within ±0.5% of 97° C.

Embodiment 59. The crystalline form of embodiment 53, wherein the crystalline form has an endothermic DSC peak temperature within ±2% of 162° C.

Embodiment 60. The crystalline form of embodiment 59, wherein the endothermic DSC peak temperature is within ±1% of 162° C.

Embodiment 61. The crystalline form of embodiment 59, wherein the endothermic DSC peak temperature is within ±0.5% of 162° C.

Embodiment 62. The crystalline form of embodiment 53, wherein the crystalline form has endothermic DSC peak temperatures within ±2% of 15° C., within ±2% of 97° C., and within ±2% of 162° C.

Embodiment 63. A crystalline form of 2-(4-(4-(aminomethyl)-1-oxo-1,2-dihydrophthalazin-6-yl)-1-methyl-1 h-pyrazol-5-yl)-4-chloro-6-cyclopropoxy-3-fluorobenzonitrile hydrochloride salt having an X-ray powder diffraction (XRPD) pattern comprising a peak at a two-theta angle of 4.3°±0.2°.

Embodiment 64. The crystalline form of embodiment 63, wherein the crystalline form has an X-ray powder diffraction (XRPD) pattern comprising a peak at a two-theta angle of 6.3°±0.2°.

Embodiment 65. The crystalline form of embodiment 63, wherein the crystalline form has an X-ray powder diffraction (XRPD) pattern comprising a peak at a two-theta angle of 7.9°±0.2°.

Embodiment 66. The crystalline form of embodiment 63, wherein the crystalline form has an X-ray powder diffraction (XRPD) pattern comprising a peak at a two-theta angle of 18.8°±0.2°.

Embodiment 67. The crystalline form of embodiment 63, wherein the crystalline form has an X-ray powder diffraction (XRPD) pattern comprising a peak at a two-theta angle of 20.3°±0.2°.

Embodiment 68. The crystalline form of embodiment 63, wherein the crystalline form has an X-ray powder diffraction (XRPD) pattern comprising a peak at a two-theta angle of 23.7°±0.2°.

Embodiment 69. The crystalline form of embodiment 63, wherein the crystalline form has an X-ray powder diffraction (XRPD) pattern comprising a peak at a two-theta angle of 26.7°±0.2°.

Embodiment 70. The crystalline form of embodiment 63, wherein the crystalline form has an X-ray powder diffraction (XRPD) pattern comprising peaks at a two-theta angle of 4.3°±0.2°, 6.3°±0.2°, 7.9°±0.2°, 18.8°±0.2°, 20.3°±0.2°, 23.7°±0.2°, and 26.7°±0.2°.

Embodiment 71. A crystalline form of 2-(4-(4-(aminomethyl)-1-oxo-1,2-dihydrophthalazin-6-yl)-1-methyl-1h-pyrazol-5-yl)-4-chloro-6-cyclopropoxy-3-fluorobenzonitrile hydrochloride salt having an X-ray powder diffraction pattern substantially shown in FIG. 4B.

Embodiment 72. The crystalline form of any of embodiments 53-71, wherein the crystalline form is a n-methyl-2-pyrrolidone (NMP) solvate.

Embodiment 73. The crystalline form of any of embodiments 53-72, wherein the crystalline form has a thermal gravimetric analysis (TGA) plot comprising a mass low of about 9.5% when heated from about 25° C. to about 150° C.

Embodiment 74. The crystalline form of any of embodiments 53-73, wherein the crystalline form has a purity of at least 97% by weight of 2-(4-(4-(aminomethyl)-1-oxo-1,2-dihydrophthalazin-6-yl)-1-methyl-1h-pyrazol-5-yl)-4-chloro-6-cyclopropoxy-3-fluorobenzonitrile hydrochloride salt.

Embodiment 75. The crystalline form of any of embodiments 53-74, wherein the crystalline form has a purity of at least 98% by weight of 2-(4-(4-(aminomethyl)-1-oxo-1,2-dihydrophthalazin-6-yl)-1-methyl-1h-pyrazol-5-yl)-4-chloro-6-cyclopropoxy-3-fluorobenzonitrile hydrochloride salt.

Embodiment 76. A pharmaceutical composition comprising the crystalline form of any of embodiments 53-75 and a pharmaceutically acceptable carrier.

Embodiment 77. A crystalline form of 2-(4-(4-(aminomethyl)-1-oxo-1,2-dihydrophthalazin-6-yl)-1-methyl-1h-pyrazol-5-yl)-4-chloro-6-cyclopropoxy-3-fluorobenzonitrile hydrochloride salt having an endothermic differential scanning calorimetric (DSC) peak temperature within ±2% of 62° C.

Embodiment 78. The crystalline form of embodiment 77, wherein the endothermic DSC peak temperature is within ±1% of 62° C.

Embodiment 79. The crystalline form of embodiment 77, wherein the endothermic DSC peak temperature is within ±0.5% of 62° C.

Embodiment 80. A crystalline form of 2-(4-(4-(aminomethyl)-1-oxo-1,2-dihydrophthalazin-6-yl)-1-methyl-1h-pyrazol-5-yl)-4-chloro-6-cyclopropoxy-3-fluorobenzonitrile hydrochloride salt having an X-ray powder diffraction (XRPD) pattern comprising a peak at a two-theta angle of 6.3°±0.2°.

Embodiment 81. The crystalline form of embodiment 80, wherein the crystalline form has an X-ray powder diffraction (XRPD) pattern comprising a peak at a two-theta angle of 18.8°±0.2°.

Embodiment 82. The crystalline form of embodiment 80, wherein the crystalline form has an X-ray powder diffraction (XRPD) pattern comprising a peak at a two-theta angle of 20.3°±0.2°.

Embodiment 83. The crystalline form of embodiment 80, wherein the crystalline form has an X-ray powder diffraction (XRPD) pattern comprising a peak at a two-theta angle of 22.3°±0.2°.

Embodiment 84. The crystalline form of embodiment 80, wherein the crystalline form has an X-ray powder diffraction (XRPD) pattern comprising a peak at a two-theta angle of 23.8°±0.2°.

Embodiment 85. The crystalline form of embodiment 80, wherein the crystalline form has an X-ray powder diffraction (XRPD) pattern comprising a peak at a two-theta angle of 26.7°±0.2°.

Embodiment 86. The crystalline form of embodiment 80, wherein the crystalline form has an X-ray powder diffraction (XRPD) pattern comprising peaks at a two-theta angle of 6.3°±0.2°, 18.8°±0.2°, 20.3°±0.2°, 22.3°±0.2°, 23.8°±0.2°, and 26.7°±0.2°.

Embodiment 87. A crystalline form of 2-(4-(4-(aminomethyl)-1-oxo-1,2-dihydrophthalazin-6-yl)-1-methyl-1h-pyrazol-5-yl)-4-chloro-6-cyclopropoxy-3-fluorobenzonitrile hydrochloride salt having an X-ray powder diffraction pattern substantially shown in FIG. 5B.

Embodiment 88. The crystalline form of any of embodiments 77-87, wherein the crystalline form is a n-methyl-2-pyrrolidone (NMP) solvate.

Embodiment 89. The crystalline form of any of embodiments 77-88, wherein the crystalline form has a thermal gravimetric analysis (TGA) plot comprising a mass loss of about 5.5% when heated from about 25° C. to about 150° C.

Embodiment 90. The crystalline form of any of embodiments 77-89, wherein the crystalline form has a purity of at least 97% by weight of 2-(4-(4-(aminomethyl)-1-oxo-1,2-dihydrophthalazin-6-yl)-1-methyl-1h-pyrazol-5-yl)-4-chloro-6-cyclopropoxy-3-fluorobenzonitrile hydrochloride salt.

Embodiment 91. The crystalline form of any of embodiments 77-90, wherein the crystalline form has a purity of at least 98% by weight of 2-(4-(4-(aminomethyl)-1-oxo-1,2-dihydrophthalazin-6-yl)-1-methyl-1h-pyrazol-5-yl)-4-chloro-6-cyclopropoxy-3-fluorobenzonitrile hydrochloride salt.

Embodiment 92. A pharmaceutical composition comprising the crystalline form of any of embodiments 77-91 and a pharmaceutically acceptable carrier.

Embodiment 93. A method for treating cancer in a subject in need thereof, comprising administering to the subject a crystalline form of a crystalline form of any one of embodiments 1-17, 19-35, 37-51, 53-75 or 77-91, or a pharmaceutical composition comprising a crystalline form of any one of embodiments 18, 36, 52, 76 or 92.

Embodiment 94. A method according to Embodiment 93 wherein the cancer is a MTAP-associated cancer.

Embodiment 95. A method according to Embodiment 93 wherein the cancer is selected from the group consisting of ovarian serous cystadenocarcinoma, squamous cell lung cancer, lung adenocarcinoma, mesothelioma; esophogeal squamous cell carcinoma, gastric adenocarcinoma, pancreatic ductal adenocarcinoma, kidney adenocarcinoma, bladder transitional cell carcinoma, hepatocellular carcinoma, cholangiocarcinoma, cholangiocarcinoma; osteosarcoma, multiple myeloma, astrocytoma, glioma, glioblastoma, uterine sarcoma, acute myeloid leukemia, acute lymphoblastic leukemia, non-Hodgkin's lymphoma, malignant melanoma, endometrial carcinoma and thyroid carcinoma.

Embodiment 96. A method according to Embodiment 93 wherein the cancer is selected from the group consisting of bladder cancer, bone cancer, brain cancer, blood cancer, breast cancer, colorectal cancer, esophageal cancer, gastric cancer, head and neck cancer, kidney cancer, liver cancer, lung cancer, mesothelioma, ovarian cancer, pancreatic cancer, skin cancer, thyroid cancer and uterine cancer

While the invention has been described in connection with specific embodiments thereof, it will be understood that it is capable of further modifications and this application is intended to cover any variations, uses, or adaptations of the invention following, in general, the principles of the invention and including such departures from this disclosure as come within known or customary practice within the art to which the invention pertains and as may be applied to the essential features hereinbefore set forth, and as follows in the scope of the appended claims.

We claim:

1. A crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile hydrochloride salt having an endothermic differential scanning calorimetric (DSC) peak temperature within ±2% of 118° C.

2. The crystalline form of claim 1, wherein the endothermic DSC peak temperature is within ±1% of 118° C.

3. The crystalline form of claim 1, wherein the endothermic DSC peak temperatures is within ±0.5% of 118° C.

4. The crystalline form of claim 1, wherein the crystalline form has an endothermic DSC peak temperature within ±2% of 211° C.

5. The crystalline form of claim 4, wherein the endothermic DSC peak temperature is within ±1% of 211° C.

6. The crystalline form of claim 4, wherein the endothermic DSC peak temperature is within ±0.5% of 211° C.

7. A crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile hydrochloride salt having an X-ray powder diffraction (XRPD) pattern comprising a peak at a two-theta angle of 6.80±0.2°.

8. The crystalline form of claim 7, wherein the crystalline form has an X-ray powder diffraction (XRPD) pattern comprising a peak at a two-theta angle of 7.4°±0.2°.

9. The crystalline form of claim 7, wherein the crystalline form has an X-ray powder diffraction (XRPD) pattern comprising a peak at a two-theta angle of 13.9±0.2°.

10. The crystalline form of claim 7, wherein the crystalline form has an X-ray powder diffraction (XRPD) pattern comprising a peak at a two-theta angle of 23.9°±0.2°.

11. The crystalline form of claim 7, wherein the crystalline form has an X-ray powder diffraction (XRPD) pattern comprising a peak at a two-theta angle of 26.2°±0.2°.

12. The crystalline form of claim 7, wherein the crystalline form has an X-ray powder diffraction (XRPD) pattern comprising peaks at a two-theta angle of 6.8°±0.2°, 7.4°±0.2°, 13.9°±0.2°, 23.9°±0.2°, and 26.2°±0.2°.

13. A crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile hydrochloride salt having an X-ray powder diffraction pattern substantially shown in FIG. 1B.

14. The crystalline form of any of claims 1-13, wherein the crystalline form is a tri-hydrate.

15. The crystalline forma of any of claims 1-14, wherein the crystalline form has a thermal gravimetric analysis (TGA) plot comprising a mass loss of about 9.7% when heated from about 25° C. to about 150° C.

16. The crystalline form of any of claims 1-15, wherein the crystalline form has a purity of at least 97% by weight of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile hydrochloride salt.

17. The crystalline form of any of claims 1-16, wherein the crystalline form has a purity of at least 98% by weight of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile hydrochloride salt.

18. A pharmaceutical composition comprising the crystalline form of any of claims 1-17 and a pharmaceutically acceptable carrier.

19. A crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile hydrochloride salt having an endothermic differential scanning calorimetric (DSC) peak temperature within ±2% of 97° C.

20. The crystalline form of claim 19, wherein the endothermic DCS peak temperature is within ±1% of 97° C.

21. The crystalline form of claim 19, wherein the endothermic DCS peak temperature is within ±0.5% of 97° C.

22. The crystalline form of claim 19, wherein the endothermic crystalline form has a DSC peak temperature within ±2% of 245° C.

23. The crystalline form of claim 19, wherein the endothermic DCS peak temperature is within ±1% of 245° C.

24. The crystalline form of claim 19, wherein the endothermic DCS peak temperature is within ±0.5% of 245° C.

25. A crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile hydrochloride salt having an X-ray powder diffraction (XRPD) pattern comprising a peak at a two-theta angle of 12.2°±0.2°.

26. The crystalline form of claim 25, wherein the crystalline form has an X-ray powder diffraction (XRPD) pattern comprising a peak at a two-theta angle of 14.3°±0.2°.

27. The crystalline form of claim 25, wherein the crystalline form has an X-ray powder diffraction (XRPD) pattern comprising a peak at a two-theta angle of 18.9°±0.2°.

28. The crystalline form of claim 25, wherein the crystalline form has an X-ray powder diffraction (XRPD) pattern comprising a peak at a two-theta angle of 23.2°±0.2°.

29. The crystalline form of claim 25, wherein the crystalline form has an X-ray powder diffraction (XRPD) pattern comprising a peak at a two-theta angle of 26.0°±0.2°.

30. The crystalline form of claim 25, wherein the crystalline form has an X-ray powder diffraction (XRPD) pattern comprising peaks at a two-theta angle of 12.2°±0.2°, 14.3°±0.20, 18.9°±0.2°, 23.2°±0.2°, and 26.0°±0.2°.

31. A crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile hydrochloride salt having an X-ray powder diffraction pattern substantially shown in FIG. 2B.

32. The crystalline form of any of claims 19-31, wherein the crystalline form is a mono-hydrate.

33. The crystalline form of any of claims 19-32, wherein the crystalline form has a thermal gravimetric analysis (TGA) plot comprising a mass loss of 3.6% when heated from about 25° C. to about 150° C.

34. The crystalline form of any of claims 19-33, wherein the crystalline form has a purity of at least 97% by weight of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile hydrochloride salt.

35. The crystalline form of any of claims 19-34, wherein the crystalline form has a purity of at least 98% by weight of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile hydrochloride salt.

36. A pharmaceutical composition comprising the crystalline form of any of claims 19-35 and a pharmaceutically acceptable carrier.

37. A crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile hydrochloride salt having an endothermic differential scanning calorimetric (DSC) peak temperature within ±2% of 239° C.

38. The crystalline form of claim 37, wherein the endothermic DSC peak temperature is within ±1% of 239° C.

39. The crystalline form of claim 37, wherein the endothermic DSC peak temperature is within ±0.5% of 239° C.

40. A crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile hydrochloride salt having an X-ray powder diffraction (XRPD) pattern comprising a peak at a two-theta angle of 9.7°±0.20.

41. The crystalline form of claim 40, wherein the crystalline form has an X-ray powder diffraction (XRPD) pattern comprising a peak at a two-theta angle of 12.1°±0.2°.

42. The crystalline form of claim 40, wherein the crystalline form has an X-ray powder diffraction (XRPD) pattern comprising a peak at a two-theta angle of 14.3°±0.2°.

43. The crystalline form of claim 40, wherein the crystalline form has an X-ray powder diffraction (XRPD) pattern comprising a peak at a two-theta angle of 22.3°±0.2°.

44. The crystalline form of claim 40, wherein the crystalline form has an X-ray powder diffraction (XRPD) pattern comprising a peak at a two-theta angle of 22.8°±0.2°.

45. The crystalline form of claim 40, wherein the crystalline form has an X-ray powder diffraction (XRPD) pattern comprising a peak at a two-theta angle of 24.5°±0.2°.

46. The crystalline form of claim 40, wherein the crystalline form has an X-ray powder diffraction (XRPD) pattern comprising peaks at a two-theta angle of 9.7°±0.2°, 12.1°±0.2°, 14.3°±0.2°, 22.3°±0.2°, 22.8°±0.2°, and 24.5°±0.2°.

47. A crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile hydrochloride salt having an X-ray powder diffraction pattern substantially shown in FIG. 3B.

48. The crystalline form of any of claims 37-47, wherein the crystalline form is an anhydrate.

49. The crystalline form of any of claims 37-48, wherein the crystalline form has a thermal gravimetric analysis (TGA) plot comprising a mass loss of about 1.0% when heated from about 25° C. to about 150° C.

50. The crystalline form of any of claims 37-49, wherein the crystalline form has a purity of at least 97% by weight of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile hydrochloride salt.

51. The crystalline form of any of claims 37-50, wherein the crystalline form has a purity of at least 98% by weight of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile hydrochloride salt.

52. A pharmaceutical composition comprising the crystalline form of any of claims 37-51 and a pharmaceutically acceptable carrier.

53. A crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile hydrochloride salt having an endothermic differential scanning calorimetric (DSC) peak temperature within ±2% of 15° C.

54. The crystalline form of claim 53, wherein the endothermic DSC peak temperature is within ±1% of 15° C.

55. The crystalline form of claim 53, wherein the endothermic DSC peak temperature is within ±0.5% of 15° C.

56. The crystalline form of claim 53, wherein the crystalline form has an endothermic DSC peak temperature within ±2% of 97° C.

57. The crystalline form of claim 56, wherein the endothermic DSC peak temperature is within ±1% of 97° C.

58. The crystalline form of claim 56, wherein the endothermic DSC peak temperature is within ±0.5% of 97° C.

59. The crystalline form of claim 53, wherein the crystalline form has an endothermic DSC peak temperature within ±2% of 162° C.

60. The crystalline form of claim 59, wherein the endothermic DSC peak temperature is within ±1% of 162° C.

61. The crystalline form of claim 59, wherein the endothermic DSC peak temperature is within ±0.5% of 162° C.

62. The crystalline form of claim 53, wherein the crystalline form has endothermic DSC peak temperatures within ±2% of 15° C., within ±2% of 97° C., and within ±2% of 162° C.

63. A crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile hydrochloride salt having an X-ray powder diffraction (XRPD) pattern comprising a peak at a two-theta angle of 4.3°±0.20.

64. The crystalline form of claim 63, wherein the crystalline form has an X-ray powder diffraction (XRPD) pattern comprising a peak at a two-theta angle of 6.3°±0.2°.

65. The crystalline form of claim 63, wherein the crystalline form has an X-ray powder diffraction (XRPD) pattern comprising a peak at a two-theta angle of 7.9°±0.2°.

66. The crystalline form of claim 63, wherein the crystalline form has an X-ray powder diffraction (XRPD) pattern comprising a peak at a two-theta angle of 18.8°±0.2°.

67. The crystalline form of claim 63, wherein the crystalline form has an X-ray powder diffraction (XRPD) pattern comprising a peak at a two-theta angle of 20.3°±0.2°.

68. The crystalline form of claim 63, wherein the crystalline form has an X-ray powder diffraction (XRPD) pattern comprising a peak at a two-theta angle of 23.7°±0.2°.

69. The crystalline form of claim 63, wherein the crystalline form has an X-ray powder diffraction (XRPD) pattern comprising a peak at a two-theta angle of 26.7°±0.2°.

70. The crystalline form of claim 63, wherein the crystalline form has an X-ray powder diffraction (XRPD) pattern comprising peaks at a two-theta angle of 4.3°±0.2°, 6.3°±0.2°, 7.9°±0.2°, 18.8°±0.2°, 20.3°±0.2°, 23.7°±0.2°, and 26.7°±0.2°.

71. A crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile hydrochloride salt having an X-ray powder diffraction pattern substantially shown in FIG. 4B.

72. The crystalline form of any of claims 53-71, wherein the crystalline form is a n-methyl-2-pyrrolidone (NMP) solvate.

73. The crystalline form of any of claims 53-72, wherein the crystalline form has a thermal gravimetric analysis (TGA) plot comprising a mass low of about 9.5% when heated from about 25° C. to about 150° C.

74. The crystalline form of any of claims 53-73, wherein the crystalline form has a purity of at least 97% by weight of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile hydrochloride salt.

75. The crystalline form of any of claims 53-74, wherein the crystalline form has a purity of at least 98% by weight of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile hydrochloride salt.

76. A pharmaceutical composition comprising the crystalline form of any of claims 53-75 and a pharmaceutically acceptable carrier.

77. A crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile hydrochloride salt having an endothermic differential scanning calorimetric (DSC) peak temperature within ±2% of 62° C.

78. The crystalline form of claim 77, wherein the endothermic DSC peak temperature is within ±1% of 62° C.

79. The crystalline form of claim 77, wherein the endothermic DSC peak temperature is within ±0.5% of 62° C.

80. A crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile hydrochloride salt having an X-ray powder diffraction (XRPD) pattern comprising a peak at a two-theta angle of 6.3°±0.2°.

81. The crystalline form of claim 80, wherein the crystalline form has an X-ray powder diffraction (XRPD) pattern comprising a peak at a two-theta angle of 18.8°±0.2°.

82. The crystalline form of claim 80, wherein the crystalline form has an X-ray powder diffraction (XRPD) pattern comprising a peak at a two-theta angle of 20.3°±0.2°.

83. The crystalline form of claim 80, wherein the crystalline form has an X-ray powder diffraction (XRPD) pattern comprising a peak at a two-theta angle of 22.3°±0.2°.

84. The crystalline form of claim 80, wherein the crystalline form has an X-ray powder diffraction (XRPD) pattern comprising a peak at a two-theta angle of 23.8°±0.2°.

85. The crystalline form of claim 80, wherein the crystalline form has an X-ray powder diffraction (XRPD) pattern comprising a peak at a two-theta angle of 26.7°±0.2°.

86. The crystalline form of claim 80, wherein the crystalline form has an X-ray powder diffraction (XRPD) pattern comprising peaks at a two-theta angle of 6.3°±0.2°, 18.8°±0.2°, 20.3°±0.2°, 22.3°±0.2°, 23.8°±0.2°, and 26.7°±0.2°.

87. A crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile hydrochloride salt having an X-ray powder diffraction pattern substantially shown in FIG. 5B.

88. The crystalline form of any of claims 77-87, wherein the crystalline form is a n-methyl-2-pyrrolidone (NMP) solvate.

89. The crystalline form of any of claims 77-88, wherein the crystalline form has a thermal gravimetric analysis (TGA) plot comprising a mass loss of about 5.5% when heated from about 25° C. to about 150° C.

90. The crystalline form of any of claims 77-89, wherein the crystalline form has a purity of at least 97% by weight of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile hydrochloride salt.

91. The crystalline form of any of claims 77-90, wherein the crystalline form has a purity of at least 98% by weight of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile hydrochloride salt.

92. A pharmaceutical composition comprising the crystalline form of any of claims 77-91 and a pharmaceutically acceptable carrier.

93. A method for treating cancer in a subject in need thereof, comprising administering to the subject a crystalline form of a crystalline form of any one of claims 1-17, 19-35, 37-51, 53-75 or 77-91, or a pharmaceutical composition comprising a crystalline form of any one of claims 18, 36, 52, 76 or 92.

94. A method according to claim 93 wherein the cancer is a MTAP-associated cancer.

95. A method according to claim 93 wherein the cancer is selected from the group consisting of ovarian serous cystadenocarcinoma, squamous cell lung cancer, lung adenocarcinoma, mesothelioma; esophogeal squamous cell carcinoma, gastric adenocarcinoma, pancreatic ductal adenocarcinoma, kidney adenocarcinoma, bladder transitional cell carcinoma, hepatocellular carcinoma, cholangiocarcinoma, cholangiocarcinoma; osteosarcoma, multiple myeloma, astrocytoma, glioma, glioblastoma, uterine sarcoma, acute myeloid leukemia, acute lymphoblastic leukemia, non-Hodgkin's lymphoma, malignant melanoma, endometrial carcinoma and thyroid carcinoma.

96. A method according to claim 93 wherein the cancer is selected from the group consisting of bladder cancer, bone cancer, brain cancer, blood cancer, breast cancer, colorectal cancer, esophageal cancer, gastric cancer, head and neck cancer, kidney cancer, liver cancer, lung cancer, mesothelioma, ovarian cancer, pancreatic cancer, skin cancer, thyroid cancer and uterine cancer
